(19) United States
(12) Patent Application Publication
(10) Pub. No.: US 2016/0228589 A1
(43) Pub. Date: Aug. 11, 2016

(54) METHODS AND COMPOSITIONS FOR IMAGING DISORDERS USING POLYSPECIFIC AGENTS

(71) Applicant: SLOAN-KETTERING INSTITUTE FOR CANCER RESEARCH, New York, NY (US)

(72) Inventors: Nerissa Therese V. Villegas, New York, NY (US); Jason S. Lewis, New York, NY (US)

(21) Appl. No.: 15/022,734

(22) PCT Filed: Sep. 18, 2014
(86) PCT No.: PCT/US14/56332

§ 371 Date: Mar. 17, 2016

Related U.S. Application Data
(60) Provisional application No. 61/879,622, filed on Sep. 18, 2013.

(51) Int. Cl. A61K 51/10

(52) U.S. Cl. CPC A61K 51/1075

(57) ABSTRACT

The present invention provides compositions and methods for detecting and/or monitoring a disease state with polyspecific imaging agents. In a particular embodiment, provided methods may be used to assess efficacy of anti-receptor tyrosine kinase and/or anti-cancer treatments. In some embodiments, the present invention provides methods and compositions relating to polyspecific imaging agents that target neurological, tumor-associated and/or intratumoral markers. For example, the present invention provides compositions, including pharmaceutical compositions, comprising anti-receptor tyrosine kinase antibodies, or fragments or characteristic portions thereof. The present invention further provides various therapeutic and/or diagnostic methods of using anti-receptor tyrosine kinase antibodies and/or compositions.

### CROSS-REFERENCE TO RELATED APPLICATIONS

This application claims the benefit under 35 U.S.C. §119(e) of U.S. Provisional Patent Application Ser. No. 61/879,622, filed Sep. 18, 2013; the entirety of which is hereby incorporated by reference.

### BACKGROUND

The pharmaceutical industry faces increasing pressure to provide accurate and effective information and therapy. New imaging technologies are central to the response to this pressure, and significant strides have been made, for example, in the development of detectable moieties and systems that image them. Particular effort is directed toward imaging technologies that can support cancer therapy, for example, in hopes of improving surgical resection of tumor vs normal tumor and/or of stratifying or monitoring patient populations for those who will respond or are responding particularly well or poorly to a given therapeutic regimen.

### SUMMARY

The present invention provides new and effective imaging technologies. Among other things, the present invention recognizes the source of a problem with conventional technologies and provides the insight that imaging agents that can simultaneously target two or more relevant markers (i.e., polyspecific imaging agents), as described herein, are particularly useful in various contexts.

Among other things, the present disclosure demonstrates that imaging agents that can detect multiple disease markers simultaneously, can enable improved diagnosis, monitoring, and/or treatment of disease. In certain embodiments, use of bispecific and polyspecific imaging agents will enable improved sensitivity and specificity within a clinical setting.

The present invention specifically encompasses the recognition that immuno-PET has gained attention as a tool for diagnosis and staging of disease due to the high targeting and site-specific delivery properties of antibodies. With a vector that targets two amplified biomarkers—EGFR and HER3, the present disclosure demonstrates that a powerful imaging probe is achieved with the potential to offer enhanced tumor-to-non-tumor background and seek out tumors that may contain both or either receptor. The present disclosure specifically establishes that MEHD7945A, a two-in-one monoclonal antibody, makes an excellent vector for detecting and imaging lesions with an overexpression of EGFR and HER3 via positron emission tomography (PET).

Among other things, the present invention encompasses the recognition that one unmet need often encountered in clinical settings is the need for a companion diagnostic to a prescribed therapy. For example, in some cases, there is a need for a diagnostic that can stratify patient populations and identify those more or less likely to respond well or poorly to a particular therapeutic regimen. Alternatively or additionally, there may be a need for a diagnostic that can monitor a subject's response to therapy, so that informed decisions can be made about continuing, terminating, or modifying therapy.

The present disclosure demonstrates that a particular polyspecific imaging agent, 89Zr/64Cu-MEHD7945A can detect tumors that overexpress EGFR and HER3 receptors, and furthermore can act as a tool for providing a predictive and/or pharmacodynamic response to targeted treatment of these two receptors. Any increase or decrease in tumor uptake of this imaging probe can mark the tumor's status during and after treatment. The current standard FDA-approved PET imaging agent is FDG. FDG is a metabolic marker and does not provide a direct correlation to the EGFR and HER3 status of the tumor. The use of a polyspecific agent as described herein (e.g., a polyspecific antibody agent) to image biological events and/or components (e.g., tissues, cells, etc) will provide better specificity and direct association to receptor tyrosine kinase targeted therapies.

Embodiments of the present invention are based on the surprising discovery that monoclonal antibodies and antibody polypeptides specific to receptor tyrosine kinases can bind to intratumoral receptor tyrosine kinases or receptor tyrosine kinases otherwise closely associated with a medical condition. Tumor-associated receptor tyrosine kinases levels can provide a highly specific and quantifiable measure of receptor tyrosine kinase expression at the tumor itself. According to some embodiments of the present invention, binding of monoclonal antibodies or antibody polypeptides to tumor-associated receptor tyrosine kinases facilitates the monitoring and/or visualization of receptor tyrosine kinase signaling in situ and provides a superior measure of the efficacy of anti-cancer treatments.

Embodiments of the present invention are based on the surprising discovery that polyspecific imaging agents can bind to one or several markers associated with a disease state. In some embodiments, the invention provides imaging methods comprising administering to a subject suffering from or susceptible to a disease a poly-specific imaging agent comprising: at least a first targeting moiety that interacts specifically with a first target marker; at least a second targeting moiety that interacts specifically with a second target marker different from the first target marker; and at least one associated detectable moiety, wherein each of the first and second target markers is characterized in that its level of expression or activity is associated with a relevant disease state wherein each of the first and second target markers is characterized by correlation with the same relevant cancer state; and detecting the detectable moiety using an imaging modality.

In one aspect of the invention, each of the first and second target markers is characterized by correlation with the same relevant disease state. In other aspects, the disease can be neurodegenerative disorders, solid tumors, or cancer. In other aspects, the neurodegenerative disorder is Alzheimer's Disease, Multiple Sclerosis, Huntington's Disease, Amyotrophic lateral sclerosis, Parkinson's Disease and muscular dystrophy. In other aspects the cancer is breast, pancreatic, non-small-cell lung, head and neck, anal and brain cancer.

In one aspect of the invention, the imaging agent is bi-specific.

In one aspect of the invention, the imaging agent comprises immunological moieties. In another aspect of the invention, the first targeting moiety is selected from the group comprising: immunoglobulins, antibodies, antibody fragments, peptides, and polypeptides. In another aspect of the invention, the first target marker is selected from the group comprising: DNA, RNA, proteins, protein complexes, phosphorylated proteins, glycosylated proteins, folded proteins, and denatured proteins.

In one aspect of the invention, the second targeting moiety is selected from the group comprising: immunoglobulins, antibodies, antibody fragments, peptides, and polypeptides. In another aspect of the invention, the second target marker is selected from the group comprising: DNA, RNA, proteins, protein complexes, phosphorylated proteins, glycosylated proteins, folded proteins, and denatured proteins.

In one aspect of the invention, the first and second targeting moieties are simultaneously selected from the group comprising: immunoglobulins, antibodies, antibody fragments, peptides, and polypeptides. In another aspect of the invention, the first and second target markers are simultaneously selected from the group comprising: DNA, RNA, proteins, protein complexes, phosphorylated proteins, glycosylated proteins, folded proteins, and denatured proteins.

In another aspect of the invention, the first and second target markers are simultaneously selected from the group comprising: Anaplastic lymphoma kinase, Alpha-fetoprotein (AFP), Beta-2-microglobulin (B2M), Beta-human chorionic gonadotropin (Beta-hCG), BCR-ABL, BRAF, CA15-3/CA27.29, CA19-9, CA-125, Calcitonin, Carcinoembryonic antigen (CEA), CD20, Chromogranin A (CgA), Cytokeratin, EGFR, Estrogen receptor (ER), progesterone receptor (PR), Fibrin/fibrinogen, HE4, HER2, Immunoglobulins, KIT, KRAS , Lactate dehydrogenase, Matrix metalloproteinases (MMP), Nuclear matrix protein 22, Prostate-specific antigen (PSA), Receptor Tyrosine kinases, Thyroglobulin, Urokinase plasminogen activator (uPA), and plasminogen activator inhibitor (PAI-1).

In one aspect of the invention, the poly-specific imaging agent simultaneously targets at least two markers selected from the receptor tyrosine kinase protein family. In another aspect of the invention, the poly-specific imaging agent simultaneously targets at least two markers selected from the group comprising: EGFR, HER2, HER3, HER4, FGF1, FGF2, FGF3, FGF4, FGF5, FGF6, FGF7, FGF18, FGF21, VEGF-A, VEGF-B, VEGF-C, VEGF-D, PIGF, EphA1, EphA2, EphA3, EphA4, EphA5, EphA6, EphA7, EphA8, EphA9, EphA10, EphB1, EphB2. EphB3, EphB4, and EphB6. In another aspect of the invention, the poly-specific imaging agent simultaneously targets EGFR and HER3.

In one aspect of the invention, the relevant disease state is a state of cancer responsiveness selected from the group comprising: responsive to therapy and resistant to resistant. In another aspect of the invention, the relevant disease state is a stage of cancer selected from the group comprising: stage 0, stage I, stage II, stage III, or stage IV.

In one aspect of the invention, the imaging modality is selected from the group comprising, Positron Emission Tomography (PET), Single Photon Emission Tomography (SPECT), Computed Tomography (CT), Magnetic Resonance Imaging (MRI), Ultrasound Imaging (US), and Optical Imaging. In another aspect of the invention, the imaging modality is Positron Emission Tomography (PET).

In one aspect of the invention, the detectable moiety is selected from the group comprising: a radiolabel, a fluorophore, a fluorochrome, an optical reporter, a magnetic reporter, an X-ray reporter, an ultrasound imaging reporter or a nanoparticle reporter. In another aspect of the invention, the detectable moiety is a radiolabel selected from the group comprising a radioisotopic element selected from the group consisting: of astatine, bismuth, carbon, copper, fluorine, gallium, indium, iodine, lutetium, nitrogen, oxygen, phosphorous, rhenium, rubidium, samarium, technetium, thallium, yttrium, and zirconium. In another aspect of the invention, the radiolabel is selected from the group comprising zirconium-89 (⁸⁹Zr), iodine-124 (¹²⁴I), iodine-131 (¹³I), iodine-125 (¹²⁵I) iodine-123 (¹²³I), bismuth-212 (²¹²Bi), bismuth-213 (²¹³Bi), astatine-221 (²¹¹At), copper-67 (⁶⁷Cu), copper-64 (⁶⁴Cu), rhenium-186 (¹⁸⁶Re), rhenium-186 (¹⁸⁸Re), phosphorus-32 (³²P), samarium-153 (¹⁵³Sm), lutetium-177 (¹¹⁷Lu), technetium-99m (^{99m}Tc), gallium-67 (⁶⁷Ga), indium-111 (¹¹¹In), thallium-201 (²⁰¹Tl) carbon-11, nitrogen-13 (¹³N), oxygen-15 (¹⁵O), fluorine-18 (¹⁸F), and rubidium-82 (⁸²Ru).

In one aspect, the invention is a poly-specific imaging agent comprising: at least a first targeting moiety that interacts specifically with a first target marker; at least a second targeting moiety that interacts specifically with a second target marker different from the first target marker; and at least one associated detectable moiety.

In another aspect, the invention is a method for treating or reducing the risk of disease comprising: administering to a subject susceptible to the disease, disorder, or condition a polyspecific imaging agent.

In one aspect, the invention is a kit for detecting the expression of target markers comprising a polyspecific imaging agent.

Other features and advantages of the invention will be apparent from the following figures, definitions, detailed description and the claims.

### DESCRIPTION OF THE DRAWING

The Figures described below, that together make up the Drawing, are for illustration purposes only, not for limitation.

FIG. 1A depicts PET imaging of mice with ⁸⁹Zr-MEHD7945A, demonstrating increasing uptake in EGFR/HER3(+) BxPC3 pancreatic cancer xenografts from 4-120 h post-injection (p.i.).

FIG. 1B depicts PET imaging of mice performing a competitive inhibition assay by addition of a blocking dose of cold MEHD7945A which lowered the uptake of 89Zr-MEHD7945A in the same mouse model demonstrating specificity of the imaging probe.

FIG. 1C depicts PET imaging of mice negative for the EGFR/HER3 receptors. EGFR/HER3(−) MIA PaCa-2 tumor implants with the same probe displayed stagnant uptake <10 %ID/g at all timepoints (24-120 h p.i.), possibly due to enhanced permeation and retention of the tumor's leaky vasculature.

FIG. 2 depicts a tissue biodistribution plot of ⁸⁹Zr-MEHD7945A in mice bearing EGFR/HER3(+) BxPC3 xenografts. Uptake in the tumor started to accumulate as early as 4 h, reaching its peak at 48 h p.i. Apparent tumor retention was observed as long as 120 h p.i. Minimal accumulation was displayed in normal healthy tissues, particularly in the pancreas.

FIG. 3 depicts Ex vivo histology and autoradiography of mice treated with ⁸⁹Zr-MEHD7945A. H&E staining (left) digital autoradiograph (center) and ex-vivo fluorescence micrograph (right, green=EGFR, blue=Hoechst 33342). All data shown from a single frozen section obtained from a subcutaneous BxPC3 murine xenograft model treated with ⁸⁹Zr-MEHD7945A.

FIG. 4A depicts in vitro binding affinity curves of ⁸⁹Zr-MEHD7945A in BxPC3 cells (KRAS-wt). Two binding affinities were observed at 0.34 nM and 12.02 nM.

FIG. 4B depicts in vitro binding affinity curves of ⁸⁹Zr-MEHD7945A in AsPC-1 cells (KRAS-mutant). Two binding affinities were observed at 0.30 nM and 23.05 nM.

FIG. 4C depicts ex vivo binding analysis of ⁸⁹Zr-MEHD7945A in AsPC-1 (KRAS-mutant) excised tumors. A non-linear regression analysis and Scatchard plot of ⁸⁹Zr-MEHD7945A in AsPC-1 tumors is shown.

FIG. 4D depicts a plot of in vivo studies using ⁸⁹Zr-MEHD7945A to measure the pharmacological effects of treatment in mice.

FIG. 4E depicts PET scans of in vivo studies using ⁸⁹Zr-MEHD7945A to measure the pharmacological effects of treatment in mice.

### DEFINITIONS

Agent: The term “agent” as used herein may refer to a compound or entity of any chemical class including, for example, polypeptides, nucleic acids, saccharides, lipids, small molecules, metals, or combinations thereof. As will be clear from context, in some embodiments, an agent can be or comprise a cell or organism, or a fraction, extract, or component thereof. In some embodiments, an agent is agent is or comprises a natural product in that it is found in and/or is obtained from nature. In some embodiments, an agent is or comprises one or more entities that is man-made in that it is designed, engineered, and/or produced through action of the hand of man and/or is not found in nature. In some embodiments, an agent may be utilized in isolated or pure form; in some embodiments, an agent may be utilized in crude form. In some embodiments, potential agents are provided as collections or libraries, for example that may be screened to identify or characterize active agents within them. Some particular embodiments of agents that may be utilized in accordance with the present invention include small molecules, antibodies, antibody fragments, aptamers, siRNAs, shRNAs, DNA/RNA hybrids, antisense oligonucleotides, ribozymes, peptides, peptide mimetics, small molecules, etc. In some embodiments, an agent is or comprises a polymer. In some embodiments, an agent is not a polymer and/or is substantially free of any polymer. In some embodiments, an agent contains at least one polymeric moiety. In some embodiments, an agent lacks or is substantially free of any polymeric moiety.

Affinity: As is known in the art, “affinity” is a measure of the tightness with a particular ligand (e.g., an HA polypeptide) binds to its partner (e.g., an HA receptor). Affinities can be measured in different ways. In some embodiments, affinity is measured by a quantitative assay (e.g., glycan binding assays). In some such embodiments, binding partner concentration (e.g., HA receptor, glycan, etc.) may be fixed to be in excess of ligand (e.g., an HA polypeptide) concentration so as to mimic physiological conditions (e.g., viral HA binding to cell surface glycans). Alternatively or additionally, in some embodiments, binding partner (e.g., HA receptor, glycan, etc.) concentration and/or ligand (e.g., an HA polypeptide) concentration may be varied. In some such embodiments, affinity (e.g., binding affinity) may be compared to a reference (e.g., a wild-type HA that mediates infection of a humans) under comparable conditions (e.g., concentrations).

Amino acid: As used herein, term “amino acid,” in its broadest sense, refers to any compound and/or substance that can be incorporated into a polypeptide chain. In some embodiments, an amino acid has the general structure H2N—C(H)(R)—COOH. In some embodiments, an amino acid is a naturally occurring amino acid. In some embodiments, an amino acid is a synthetic amino acid; in some embodiments, an amino acid is a d-amino acid; in some embodiments, an amino acid is an 1-amino acid. “Standard amino acid” refers to any of the twenty standard 1-amino acids commonly found in naturally occurring peptides. “Nonstandard amino acid” refers to any amino acid, other than the standard amino acids, regardless of whether it is prepared synthetically or obtained from a natural source. As used herein, “synthetic amino acid” encompasses chemically modified amino acids, including but not limited to salts, amino acid derivatives (such as amides), and/or substitutions. Amino acids, including carboxy- and/or amino-terminal amino acids in peptides, can be modified by methylation, amidation, acetylation, protecting groups, and/or substitution with other chemical groups that can change the peptide's circulating half-life without adversely affecting their activity. Amino acids may participate in a disulfide bond. Amino acids may comprise one or posttranslational modifications, such as association with one or more chemical entities (e.g., methyl groups, acetate groups, acetyl groups, phosphate groups, formyl moieties, isoprenoid groups, sulfate groups, polyethylene glycol moieties, lipid moieties, carbohydrate moieties, biotin moieties, etc.). The term “amino acid” is used interchangeably with “amino acid residue,” and may refer to a free amino acid and/or to an amino acid residue of a peptide. It will be apparent from the context in which the term is used whether it refers to a free amino acid or a residue of a peptide.

Animal: As used herein, the term “animal” refers to any member of the animal kingdom. In some embodiments, “animal” refers to humans, of either sex and at any stage of development. In some embodiments, “animal” refers to non-human animals, at any stage of development. In certain embodiments, the non-human animal is a mammal (e.g., a rodent, a mouse, a rat, a rabbit, a monkey, a dog, a cat, a sheep, cattle, a primate, and/or a pig). In some embodiments, animals include, but are not limited to, mammals, birds, reptiles, amphibians, fish, insects, and/or worms. In certain embodiments, the animal is susceptible to infection by HCV. In some embodiments, an animal may be a transgenic animal, genetically engineered animal, and/or a clone.

Antibody agent: As used herein, the term “antibody agent” refers to an agent that specifically binds to a particular antigen. In some embodiments, the term encompasses any polypeptide with immunoglobulin structural elements sufficient to confer specific binding. Suitable antibody agents include, but are not limited to, human antibodies, primatized antibodies, chimeric antibodies, bi-specific antibodies, humanized antibodies, conjugated antibodies (i.e., antibodies conjugated or fused to other proteins, radiolabels, cytotoxins), Small Modular ImmunoPharmaceuticals (“SMIPs™”), single chain antibodies, cameloid antibodies, and antibody fragments. As used herein, the term “antibody agent” also includes intact monoclonal antibodies, polyclonal antibodies, single domain antibodies (e.g., shark single domain antibodies (e.g., IgNAR or fragments thereof)), multispecific antibodies (e.g. bi-specific antibodies) formed from at least two intact antibodies, and antibody fragments so long as they exhibit the desired biological activity. In some embodiments, the term encompasses stapled peptides. In some embodiments, the term encompasses one or more antibody-like binding peptidomimetics. In some embodiments, the term encompasses one or more antibody-like binding scaffold proteins. In come embodiments, the term encompasses monobodies or adnectins. In many embodiments, an antibody agent is or comprises a polypeptide whose amino acid sequence includes one or more structural elements recognized by those skilled in the art as a complementarity determining region (CDR); in some embodiments an antibody agent is or comprises a polypeptide whose amino acid sequence includes at least one CDR (e.g., at least one heavy chain CDR and/or at least one light chain CDR) that is substantially identical to one found in a reference antibody. In some embodiments an included CDR is substantially identical to a reference CDR in that it is either identical in sequence or contains between 1-5 amino acid substitutions as compared with the reference CDR. In some embodiments an included CDR is substantially identical to a reference CDR in that it shows at least 85%, 86%, 87%, 88%, 89%, 90%, 91%, 92%, 93%, 94%, 95%, 96%, 97%, 98%, 99%, or 100% sequence identity with the reference CDR. In some embodiments an included CDR is substantially identical to a reference CDR in that it shows at least 96%, 96%, 97%, 98%, 99%, or 100% sequence identity with the reference CDR. In some embodiments an included CDR is substantially identical to a reference CDR in that at least one amino acid within the included CDR is deleted, added, or substituted as compared with the reference CDR but the included CDR has an amino acid sequence that is otherwise identical with that of the reference CDR. In some embodiments an included CDR is substantially identical to a reference CDR in that 1-5 amino acids within the included CDR are deleted, added, or substituted as compared with the reference CDR but the included CDR has an amino acid sequence that is otherwise identical to the reference CDR . In some embodiments an included CDR is substantially identical to a reference CDR in that at least one amino acid within the included CDR is substituted as compared with the reference CDR but the included CDR has an amino acid sequence that is otherwise identical with that of the reference CDR. In some embodiments an included CDR is substantially identical to a reference CDR in that 1-5 amino acids within the included CDR are deleted, added, or substituted as compared with the reference CDR but the included CDR has an amino acid sequence that is otherwise identical to the reference CDR. In some embodiments, an antibody agent is or comprises a polypeptide whose amino acid sequence includes structural elements recognized by those skilled in the art as an immunoglobulin variable domain. In some embodiments, an antibody agent is a polypeptide protein having a binding domain which is homologous or largely homologous to an immunoglobulin-binding domain.

Antagonist: As used herein, the term “antagonist” refers to an agent that i) inhibits, decreases or reduces the effects of another agent, for example that inactivates a receptor; and/or ii) inhibits, decreases, reduces, or delays one or more biological events, for example, activation of one or more receptors or stimulation of one or more biological pathways. In particular embodiments, an antagonist inhibits activation and/or activity of one or more receptor tyrosine kinases. Antagonists may be or include agents of any chemical class including, for example, small molecules, polypeptides, nucleic acids, carbohydrates, lipids, metals, and/or any other entity that shows the relevant inhibitory activity. An antagonist may be direct (in which case it experts its influence directly upon the receptor) or indirect (in which case it exerts its influence by other than binding to the receptor; e.g., altering expression or translation of the receptor; altering signal transduction pathways that are directly activated by the receptor, altering expression, translation or activity of an agonist of the receptor). In particular embodiments, receptor tyrosine kinase antagonists may be selected from the group consisting of small molecule antagonists (e.g., RU58642, LG120907, LG105, RD162, MDV3100, BMS-641988, CH5137291, ataric acid, N-butylbenzenesulfonamide), steroidal compounds (e.g., cyproterone acetate), non-steroidal compounds (e.g., hydroxyflutamide, bicalutamide, nilutamide), peptide antagonists, and combinations thereof Alternatively or additionally, anti-receptor tyrosine kinase therapies for use in embodiments of the invention include, but are not limited to TAK700, ARN-509, cabozantimib, ipilimumab, custirsen, BPX-101, alpharadin, denosumab, Protsvac-VF, and combinations thereof

Antibody polypeptide: As used herein, the terms “antibody polypeptide” or “antibody”, or “antigen-binding fragment thereof”, which may be used interchangeably, refer to polypeptide(s) capable of binding to an epitope. In some embodiments, an antibody polypeptide is a full-length antibody, and in some embodiments, is less than full length but includes at least one binding site (comprising at least one, and preferably at least two sequences with structure of antibody “variable regions”). In some embodiments, the term “antibody polypeptide” encompasses any protein having a binding domain which is homologous or largely homologous to an immunoglobulin-binding domain. In particular embodiments, “antibody polypeptides” encompasses polypeptides having a binding domain that shows at least 99% identity with an immunoglobulin binding domain. In some embodiments, “antibody polypeptide” is any protein having a binding domain that shows at least 70%, 80%, 85%, 90%, or 95% identity with an immuglobulin binding domain, for example a reference immunoglobulin binding domain. An included “antibody polypeptide” may have an amino acid sequence identical to that of an antibody that is found in a natural source. Antibody polypeptides in accordance with the present invention may be prepared by any available means including, for example, isolation from a natural source or antibody library, recombinant production in or with a host system, chemical synthesis, etc., or combinations thereof. An antibody polypeptide may be monoclonal or polyclonal. An antibody polypeptide may be a member of any immunoglobulin class, including any of the human classes: IgG, IgM, IgA, IgD, and IgE. In certain embodiments, an antibody may be a member of the IgG immunoglobulin class. As used herein, the terms “antibody polypeptide” or “characteristic portion of an antibody” are used interchangeably and refer to any derivative of an antibody that possesses the ability to bind to an epitope of interest. In certain embodiments, the “antibody polypeptide” is an antibody fragment that retains at least a significant portion of the full-length antibody's specific binding ability. Examples of antibody fragments include, but are not limited to, Fab, Fab′, F(ab′)2, scFv, Fv, dsFv diabody, and Fd fragments. Alternatively or additionally, an antibody fragment may comprise multiple chains that are linked together, for example, by disulfide linkages. In some embodiments, an antibody polypeptide may be a human antibody. In some embodiments, the antibody polypeptides may be a humanized. Humanized antibody polypeptides include may be chimeric immunoglobulins, immunoglobulin chains or antibody polypeptides (such as Fv, Fab, Fab′, F(ab′)2 or other antigen-binding subsequences of antibodies) that contain minimal sequence derived from non-human immunoglobulin. In general, humanized antibodies are human immunoglobulins (recipient antibody) in which residues from a complementary-determining region (CDR) of the recipient are replaced by residues from a CDR of a non-human species (donor antibody) such as mouse, rat or rabbit having the desired specificity, affinity, and capacity. In particular embodiments, antibody polyeptides for use in accordance with the present invention bind to particular epitopes of receptor tyrosine kinases (for example, in the catalytic cleft); in some embodiments, antibody polypeptides for use in accordance with the present invention are specific for particular epitopes of receptor tyrosine kinases.

Antigen: An “antigen” is a molecule or entity to which an antibody binds. In some embodiments, an antigen is or comprises a polypeptide or portion thereof In some embodiments, an antigen is a portion of an infectious agent that is recognized by antibodies. In some embodiments, an antigen is an agent that elicits an immune response; and/or (ii) an agent that is bound by a T cell receptor (e.g., when presented by an MHC molecule) or to an antibody (e.g., produced by a B cell) when exposed or administered to an organism. In some embodiments, an antigen elicits a humoral response (e.g., including production of antigen-specific antibodies) in an organism; alternatively or additionally, in some embodiments, an antigen elicits a cellular response (e.g., involving T-cells whose receptors specifically interact with the antigen) in an organism. It will be appreciated by those skilled in the art that a particular antigen may elicit an immune response in one or several members of a target organism (e.g., mice, rabbits, primates, humans), but not in all members of the target organism species. In some embodiments, an antigen elicits an immune response in at least about 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% of the members of a target organism species. In some embodiments, an antigen binds to an antibody and/or T cell receptor, and may or may not induce a particular physiological response in an organism. In some embodiments, for example, an antigen may bind to an antibody and/or to a T cell receptor in vitro, whether or not such an interaction occurs in vivo. In general, an antigen may be or include any chemical entity such as, for example, a small molecule, a nucleic acid, a polypeptide, a carbohydrate, a lipid, a polymer [in some embodiments other than a biologic polymer (e.g., other than a nucleic acid or amino acid polymer)] etc. In some embodiments, an antigen is or comprises a polypeptide. In some embodiments, an antigen is or comprises a glycan. Those of ordinary skill in the art will appreciate that, in general, an antigen may be provided in isolated or pure form, or alternatively may be provided in crude form (e.g., together with other materials, for example in an extract such as a cellular extract or other relatively crude preparation of an antigen-containing source). In some embodiments, antigens utilized in accordance with the present invention are provided in a crude form. In some embodiments, an antigen is or comprises a recombinant antigen.

Approximately: As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Biologically active: As used herein, the phrase “biologically active” refers to a characteristic of any substance that has activity in a biological system (e.g., cell culture, organism, etc.). For instance, a substance that, when administered to an organism, has a biological effect on that organism, is considered to be biologically active. In particular embodiments, where a protein or polypeptide is biologically active, a portion of that protein or polypeptide that shares at least one biological activity of the protein or polypeptide is typically referred to as a “biologically active” portion.

Characteristic portion: As used herein, the term a “characteristic portion” of a substance, in the broadest sense, is one that shares some degree of sequence or structural identity with respect to the whole substance. In certain embodiments, a characteristic portion shares at least one functional characteristic with the intact substance. For example, a “characteristic portion” of a protein or polypeptide is one that contains a continuous stretch of amino acids, or a collection of continuous stretches of amino acids, that together are characteristic of a protein or polypeptide. In some embodiments, each such continuous stretch generally contains at least 2, 5, 10, 15, 20, 50, or more amino acids. In general, a characteristic portion of a substance (e.g., of a protein, antibody, etc.) is one that, in addition to the sequence and/or structural identity specified above, shares at least one functional characteristic with the relevant intact substance; epitope-binding specificity is one example. In some embodiments, a characteristic portion may be biologically active.

Combination therapy: The term “combination therapy”, as used herein, refers to those situations in which two or more different pharmaceutical agents for the treatement of disease are administered in overlapping regimens so that the subject is simultaneously exposed to at least two agents. In some embodiments, the different agents are administered simultaneously. In some embodiments, the administration of one agent overlaps the administration of at least one other agent. In some embodiments, the different agents are administered sequentially such that the agents have simultaneous biologically activity with in a subject.

Detection entity: The term “detection entity” as used herein refers to any element, molecule, functional group, compound, fragments thereof or moiety that facilitates detection of an agent (e.g., an antibody) to which it is joined. Examples of detection entities include, but are not limited to: various ligands, radionuclides (e.g., ³H, ¹⁴C, ¹⁸F, ¹⁹F, ³²P, ³⁵S, ¹³⁵I, ¹²⁵I, ¹²³I, ⁶⁴Cu, ¹⁸⁷Re, ¹¹¹In, ⁹⁰Y, ^{99m}Tc, ¹⁷⁷Lu, ⁸⁹Zr etc.), fluorescent dyes (for specific exemplary fluorescent dyes, see below), chemiluminescent agents (such as, for example, acridinum esters, stabilized dioxetanes, and the like), bioluminescent agents, spectrally resolvable inorganic fluorescent semiconductors nanocrystals (i.e., quantum dots), metal nanoparticles (e.g., gold, silver, copper, platinum, etc.) nanoclusters, paramagnetic metal ions, enzymes (for specific examples of enzymes, see below), colorimetric labels (such as, for example, dyes, colloidal gold, and the like), biotin, dioxigenin, haptens, and proteins for which antisera or monoclonal antibodies are available.

Diagnostic information: As used herein, diagnostic information or information for use in diagnosis is any information that is useful in determining whether a patient has a disease or condition and/or in classifying the disease or condition into a phenotypic category or any category having significance with regard to prognosis of the disease or condition, or likely response to treatment (either treatment in general or any particular treatment) of the disease or condition. Similarly, diagnosis refers to providing any type of diagnostic information, including, but not limited to, whether a subject is likely to have a disease or condition (such as cancer), state, staging or characteristic of the disease or condition as manifested in the subject, information related to the nature or classification of a tumor, information related to prognosis and/or information useful in selecting an appropriate treatment. Selection of treatment may include the choice of a particular therapeutic (e.g., chemotherapeutic) agent or other treatment modalitiy such as surgery, radiation, etc., a choice about whether to withhold or deliver therapy, a choice relating to dosing regimen (e.g., frequency or level of one or more doses of a particular therapeutic agent or combination of therapeutic agents), etc.

Dosage form: As used herein, the terms “dosage form” and “unit dosage form” refer to a physically discrete unit of a therapeutic composition to be administered to a subject. Each unit contains a predetermined quantity of active material (e.g., a therapeutic agent such as an anti-receptor tyrosine kinases antibody). In some embodiments, the predetermined quantity is one that has been correlated with a desired therapeutic effect when admininstered as a dose in a dosing regimen. Those of ordinary skill in the art appreciate that the total amount of a therapeutic composition or agent administered to a particular subject is determined by one or more attending physicians and may involve administration of multiple dosage forms

Dosing regimen: A “dosing regimen” (or “therapeutic regimen”), as that term is used herein, is a set of unit doses (typically more than one) that are administered individually to a subject, typically separated by periods of time. In some embodiments, a given therapeutic agent has a recommended dosing regimen, which may involve one or more doses. In some embodiments, a dosing regimen comprises a plurality of doses each of which are separated from one another by a time period of the same length; in some embodiments, a dosing regime comprises a plurality of doses and at least two different time periods separating individual doses. In some embodiments, a dosing regimen is or has been correlated with a desired therapeutic outcome, when administered across a population of patients.

Expression: As used herein, “expression” of a nucleic acid sequence refers to one or more of the following events: (1) production of an RNA template from a DNA sequence (e.g., by transcription); (2) processing of an RNA transcript (e.g., by splicing, editing, 5′ cap formation, and/or 3′ end formation); (3) translation of an RNA into a polypeptide or protein; and/or (4) post-translational modification of a polypeptide or protein.

Functional: As used herein, a “functional” biological molecule is a biological molecule in a form in which it exhibits a property and/or activity by which it is characterized. A biological molecule may have two functions (i.e., bifunctional) or many functions (i.e., multifunctional).

Gene: As used herein, the term “gene” has its meaning as understood in the art. In some embodiments, the term “gene” may include gene regulatory sequences (e.g., promoters, enhancers, etc.) and/or intron sequences. In some embodiments, the term refers to nucleic acids that do not encode proteins but rather encode functional RNA molecules such as tRNAs, RNAi-inducing agents, etc. Alternatively or additionally, in many embodiments, the term “gene”, as used in the present application, refers to a portion of a nucleic acid that encodes a protein. Whether the term encompasses other sequences (e.g., non-coding sequences, regulatory sequences, etc) will be clear from context to those of ordinary skill in the art.

Gene product or expression product: As used herein, the term “gene product” or “expression product” generally refers to an RNA transcribed from the gene (pre-and/or post-processing) or a polypeptide (pre- and/or post-modification) encoded by an RNA transcribed from the gene.

Homology: As used herein, the term “homology” refers to the overall relatedness between polymeric molecules, e.g., between polypeptide molecules. In some embodiments, polymeric molecules such as antibodies are considered to be “homologous” to one another if their sequences are at least 80%, 85%, 90%, 95%, or 99% identical. In some embodiments, polymeric molecules are considered to be “homologous” to one another if their sequences are at least 80%, 85%, 90%, 95%, or 99% similar.

Marker: A marker, as used herein, refers to an agent whose presence or level is a characteristic of a particular tumor or metastatic disease thereof For example, in some embodiments, the term refers to a gene expression product that is characteristic of a particular tumor, tumor subclass, stage of tumor, etc. Alternatively or additionally, in some embodiments, a presence or level of a particular marker correlates with activity (or activity level) of a particular signaling pathway, for example that may be characteristic of a particular class of tumors. The statistical significance of the presence or absence of a marker may vary depending upon the particular marker. In some embodiments, detection of a marker is highly specific in that it reflects a high probability that the tumor is of a particular subclass. Such specificity may come at the cost of sensitivity (i.e., a negative result may occur even if the tumor is a tumor that would be expected to express the marker). Conversely, markers with a high degree of sensitivity may be less specific that those with lower sensitivity. According to the present invention a useful marker need not distinguish tumors of a particular subclass with 100% accuracy.

Patient: As used herein, the term “patient” or “subject” refers to any organism to which a provided composition is or may be administered, e.g., for experimental, diagnostic, prophylactic, cosmetic, and/or therapeutic purposes. Typical patients include animals (e.g., mammals such as mice, rats, rabbits, non-human primates, and/or humans). In some embodiments, a patient is a human. In some embodiments, a patient is suffering from or susceptible to one or more disorders or conditions. In some embodiments, a patient displays one or more symptoms of a disorder or condition. In some embodiments, a patient has been diagnosed with one or more disorders or conditions. In some embodiments, the disorder or condition is or includes cancer, or presence of one or more tumors. In some embodiments, such cancer or tumor is or comprises a cancer of the prostate, or tumor in the prostate. In some embodiments, the disorder or condition is metastatic cancer.

Peptide: The term “peptide” refers to two or more amino acids joined to each other by peptide bonds or modified peptide bonds. In particular embodiments, “peptide” refers to a polypeptide having a length of less than about 100 amino acids, less than about 50 amino acids, less than 20 amino acids, or less than 10 amino acids.

Pharmaceutically acceptable: The term “pharmaceutically acceptable” as used herein, refers to substances that, within the scope of sound medical judgment, are suitable for use in contact with the tissues of human beings and animals without excessive toxicity, irritation, allergic response, or other problem or complication, commensurate with a reasonable benefit/risk ratio.

Pharmaceutical composition: As used herein, the term “pharmaceutical composition” refers to an active agent, formulated together with one or more pharmaceutically acceptable carriers. In some embodiments, active agent is present in unit dose amount appropriate for administration in a therapeutic regimen that shows a statistically significant probability of achieving a predetermined therapeutic effect when administered to a relevant population. In some embodiments, pharmaceutical compositions may be specially formulated for administration in solid or liquid form, including those adapted for the following: oral administration, for example, drenches (aqueous or non-aqueous solutions or suspensions), tablets, e.g., those targeted for buccal, sublingual, and systemic absorption, boluses, powders, granules, pastes for application to the tongue; parenteral administration, for example, by subcutaneous, intramuscular, intravenous or epidural injection as, for example, a sterile solution or suspension, or sustained-release formulation; topical application, for example, as a cream, ointment, or a controlled-release patch or spray applied to the skin, lungs, or oral cavity; intravaginally or intrarectally, for example, as a pessary, cream, or foam; sublingually; ocularly; transdermally; or nasally, pulmonary, and to other mucosal surfaces.

Polypeptide: As used herein, a “polypeptide”, generally speaking, is a string of at least two amino acids attached to one another by a peptide bond. In some embodiments, a polypeptide may include at least 3-5 amino acids, each of which is attached to others by way of at least one peptide bond. Those of ordinary skill in the art will appreciate that polypeptides sometimes include “non-natural” amino acids or other entities that nonetheless are capable of integrating into a polypeptide chain, optionally.

Prognostic and predictive information: As used herein, the terms prognostic and predictive information are used interchangeably to refer to any information that may be used to indicate any aspect of the course of a disease or condition either in the absence or presence of treatment. Such information may include, but is not limited to, the average life expectancy of a patient, the likelihood that a patient will survive for a given amount of time (e.g., 6 months, 1 year, 5 years, etc.), the likelihood that a patient will be cured of a disease, the likelihood that a patient's disease will respond to a particular therapy (wherein response may be defined in any of a variety of ways). Prognostic and predictive information are included within the broad category of diagnostic information.

Protein: As used herein, the term “protein” refers to a polypeptide (i.e., a string of at least 3-5 amino acids linked to one another by peptide bonds). Proteins may include moieties other than amino acids (e.g., may be glycoproteins, proteoglycans, etc.) and/or may be otherwise processed or modified. In some embodiments “protein” can be a complete polypeptide as produced by and/or active in a cell (with or without a signal sequence); in some embodiments, a “protein” is or comprises a characteristic portion such as a polypeptide as produced by and/or active in a cell. In some embodiments, a protein includes more than one polypeptide chain. For example, polypeptide chains may be linked by one or more disulfide bonds or associated by other means. In some embodiments, proteins or polypeptides as described herein may contain L-amino acids, D-amino acids, or both, and/or may contain any of a variety of amino acid modifications or analogs known in the art. Useful modifications include, e.g., terminal acetylation, amidation, methylation, etc. In some embodiments, proteins or polypeptides may comprise natural amino acids, non-natural amino acids, synthetic amino acids, and/or combinations thereof. In some embodiments, proteins are or comprise antibodies, antibody polypeptides, antibody fragments, biologically active portions thereof, and/or characteristic portions thereof

Receptor tyrosine kinase: The term “receptor tyrosine kinase”, as used herein, refers to any members of the protein family of receptor tyrosine kinases (RTK), which includes but is not limited to sub-families such as Epidermal Growth Factor Receptors (EGFR) (including ErbB1/EGFR, ErbB2/HER2, ErbB3/HER3, and ErbB4/HER4), Fibroblast Growth Factor Receptors (FGFR) (including FGF1, FGF2, FGF3, FGF4, FGF5, FGF6, FGF7, FGF18, and FGF21) Vascular Endothelial Growth Factor Receptors (VEGFR) (including VEGF-A, VEGF-B, VEGF-C, VEGF-D, and PIGF), RET Receptor and the Eph Receptor Family (including EphA1, EphA2, EphA3, EphA4, EphA5, EphA6, EphA7, EphA8, EphA9, EphA10, EphB1, EphB2. EphB3, EphB4, and EphB6).

Response: As used herein, a response to treatment may refer to any beneficial alteration in a subject's condition that occurs as a result of or correlates with treatment. Such alteration may include stabilization of the condition (e.g., prevention of deterioration that would have taken place in the absence of the treatment), amelioration of symptoms of the condition, and/or improvement in the prospects for cure of the condition, etc. It may refer to a subject's response or to a tumor's response. Tumor or subject response may be measured according to a wide variety of criteria, including clinical criteria and objective criteria. Techniques for assessing response include, but are not limited to, clinical examination, positron emission tomatography, chest X-ray CT scan, MRI, ultrasound, endoscopy, laparoscopy, presence or level of tumor markers in a sample obtained from a subject, cytology, and/or histology. Many of these techniques attempt to determine the size of a tumor or otherwise determine the total tumor burden. Methods and guidelines for assessing response to treatment are discussed in Therasse et. al., “New guidelines to evaluate the response to treatment in solid tumors”, European Organization for Research and Treatment of Cancer, National Cancer Institute of the United States, National Cancer Institute of Canada, J. Natl. Cancer Inst., 2000, 92(3):205-216. The exact response criteria can be selected in any appropriate manner, provided that when comparing groups of tumors and/or patients, the groups to be compared are assessed based on the same or comparable criteria for determining response rate. One of ordinary skill in the art will be able to select appropriate criteria.

Sample: As used herein, a sample obtained from a subject may include, but is not limited to, any or all of the following: a cell or cells, a portion of tissue, blood, serum, ascites, urine, saliva, and other body fluids, secretions, or excretions. The term “sample” also includes any material derived by processing such a sample. Derived samples may include nucleotide molecules or polypeptides extracted from the sample or obtained by subjecting the sample to techniques such as amplification or reverse transcription of mRNA, etc.

Specific binding: As used herein, the terms “specific binding” or “specific for” or “specific to” refer to an interaction (typically non-covalent) between a target entity (e.g., a target protein or polypeptide) and a binding agent (e.g., an antibody, such as a provided antibody). As will be understood by those of ordinary skill, an interaction is considered to be “specific” if it is favored in the presence of alternative interactions. In many embodiments, an interaction is typically dependent upon the presence of a particular structural feature of the target molecule such as an antigenic determinant or epitope recognized by the binding molecule. For example, if an antibody is specific for epitope A, the presence of a polypeptide containing epitope A or the presence of free unlabeled A in a reaction containing both free labeled A and the antibody thereto, will reduce the amount of labeled A that binds to the antibody. It is to be understood that specificity need not be absolute. For example, it is well known in the art that numerous antibodies cross-react with other epitopes in addition to those present in the target molecule. Such cross-reactivity may be acceptable depending upon the application for which the antibody is to be used. In particular embodiments, an antibody specific for receptor tyrosine kinases has less than 10% cross-reactivity with receptor tyrosine kinase bound to protease inhibitors (e.g., ACT). One of ordinary skill in the art will be able to select antibodies having a sufficient degree of specificity to perform appropriately in any given application (e.g., for detection of a target molecule, for therapeutic purposes, etc.). Specificity may be evaluated in the context of additional factors such as the affinity of the binding molecule for the target molecule versus the affinity of the binding molecule for other targets (e.g., competitors). If a binding molecule exhibits a high affinity for a target molecule that it is desired to detect and low affinity for non-target molecules, the antibody will likely be an acceptable reagent for immunodiagnostic purposes. Once the specificity of a binding molecule is established in one or more contexts, it may be employed in other, preferably similar, contexts without necessarily re-evaluating its specificity.

Stage of cancer: As used herein, the term “stage of cancer” refers to a qualitative or quantitative assessment of the level of advancement of a cancer. Criteria used to determine the stage of a cancer include, but are not limited to, the size of the tumor and the extent of metastases (e.g., localized or distant).

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Suffering from: An individual who is “suffering from” a disease, disorder, or condition (cancer) has been diagnosed with and/or exhibits one or more symptoms of the disease, disorder, or condition. In some embodiments, an individual who is suffering from cancer is an individual who has increased tumor-associated or intratumoral receptor tyrosine kinases relative to an individual who does not have cancer.

Symptoms are reduced: According to the present invention, “symptoms are reduced” when one or more symptoms of a particular disease, disorder or condition is reduced in magnitude (e.g., intensity, severity, etc.) and/or frequency. For purposes of clarity, a delay in the onset of a particular symptom is considered one form of reducing the frequency of that symptom. Many cancer patients with smaller tumors have no symptoms. It is not intended that the present invention be limited only to cases where the symptoms are eliminated. The present invention specifically contemplates treatment such that one or more symptoms is/are reduced (and the condition of the subject is thereby “improved”), albeit not completely eliminated.

Therapeutic agent: As used herein, the phrase “therapeutic agent” refers to any agent that has a therapeutic effect and/or elicits a desired biological and/or pharmacological effect, when administered to a subject.

Therapeutically effective amount: As used herein, the term “therapeutically effective amount” refers to an amount of a therapeutic protein (e.g., receptor tyrosine kinases antibody) which confers a therapeutic effect on the treated subject, at a reasonable benefit/risk ratio applicable to any medical treatment. The therapeutic effect may be objective (i.e., measurable by some test or marker) or subjective (i.e., subject gives an indication of or feels an effect). In particular, the “therapeutically effective amount” refers to an amount of a therapeutic protein or composition effective to treat, ameliorate, or prevent a desired disease or condition, or to exhibit a detectable therapeutic or preventative effect, such as by ameliorating symptoms associated with the disease, preventing or delaying the onset of the disease, and/or also lessening the severity or frequency of symptoms of the disease. A therapeutically effective amount is commonly administered in a dosing regimen that may comprise multiple unit doses. For any particular therapeutic protein, a therapeutically effective amount (and/or an appropriate unit dose within an effective dosing regimen) may vary, for example, depending on route of administration, on combination with other pharmaceutical agents. Also, the specific therapeutically effective amount (and/or unit dose) for any particular patient may depend upon a variety of factors including the disorder being treated and the severity of the disorder; the activity of the specific pharmaceutical agent employed; the specific composition employed; the age, body weight, general health, sex and diet of the patient; the time of administration, route of administration, and/or rate of excretion or metabolism of the specific fusion protein employed; the duration of the treatment; and like factors as is well known in the medical arts.

Treatment: As used herein, the term “treatment” (also “treat” or “treating”) refers to any administration of a substance (e.g., anti-receptor tyrosine kinases antibodies or receptor tyrosine kinase antagonists) that partially or completely alleviates, ameliorates, relives, inhibits, delays onset of, reduces severity of, and/or reduces incidence of one or more symptoms, features, and/or causes of a particular disease, disorder, and/or condition (e.g., cancer). Such treatment may be of a subject who does not exhibit signs of the relevant disease, disorder and/or condition and/or of a subject who exhibits only early signs of the disease, disorder, and/or condition. Alternatively or additionally, such treatment may be of a subject who exhibits one or more established signs of the relevant disease, disorder and/or condition. In some embodiments, treatment may be of a subject who has been diagnosed as suffering from the relevant disease, disorder, and/or condition. In some embodiments, treatment may be of a subject known to have one or more susceptibility factors that are statistically correlated with increased risk of development of the relevant disease, disorder, and/or condition.

### DETAILED DESCRIPTION OF CERTAIN EMBODIMENTS

The present invention is based, in part, upon the discovery that it is possible to produce polyspecific imaging agents that are stable and biocompatible, and that can be used in a variety of in vivo and in vitro assays and imaging applications, as well as in a variety of therapeutic applications. In certain embodiments, the imaging agents are bispecific for members of the receptor tyrosine kinase (RTK) superfamily.

Clinical markers are frequently used for diagnosis of diseases such as cancer and neurodegenerative disorders. Such markers also enable assessments of treatment efficacy. While treating breast cancer with therapeutics specific to the EGFR protein HER2, other EGFR markers within the RTK superfamily, EGFR and HER3 are overexpressed. Dimerization of EGFR and HER3 indicates an aggressive form of cancer and the development of resistance (Chandarlapaty et al., Cancer Cell, 2011, 19: 58-71).

Embodiments of the present invention are drawn to methods of imaging conditions such as cancer and neurodegenerative disorders using polyspecific imaging agents. In some embodiments, the agent comprises a first targeting moiety that interacts specifically with a first target marker; a second targeting moiety that interacts with a second target marker different from the first target marker; and an associated detectable moiety.

In some embodiments, each of the first and second target markers is characterized in that its level of expression or activity is associated with a relevant disease state; wherein each of the first and second target markers is characterized by correlation with the same relevant disease state. In other embodiments, the disease state correlates with the type of disease (type of cancer, type of neurodegenerative disorder, responsiveness of the disease to treatment, and state of resistance of the disease to treatment). In some embodiments, the invention is used to monitor response to treatment. In other embodiments, the invention is used to measure the level of resistance to treatment generated by the disease. In some embodiments the markers correlate with resistance to a type of therapy. In some embodiments, the invention is used to monitor a stage of cancer selected from the group comprising: stage 0, stage I, stage II, stage III, or stage IV. In some embodiments, the invention is used to note the steps of changing the course of treatment in light of new data.

In some embodiments, solid tumors are imaged. In some embodiments, the type of disease imaged is cancer. In some embodiments, the cancer is selected from the breast, pancreatic, head and neck, anal, brain, and non-small-cell lung. In some embodiments, the form of brain cancer detected is glioblastoma multiforme. In other embodiments, the invention is used to image tumor vessels.

In other embodiments, the type of disease imaged is a neurodegenerative disorder. In some embodiments, the neurodegenerative disorder is selected from Alzheimer's Disease, Multiple Sclerosis, Huntington's Disease, Amyotrophic lateral sclerosis, Parkinson's Disease and muscular dystrophy.

In some embodiments, the imaging agent is bi-specific and recognizes two markers simultaneously. In other embodiments, the agent also recognizes each of the markers independently. In some embodiments, the imaging agent comprises immunological moieties able to recognize two markers simultaneously. In some embodiments, the first targeting moiety are selected from the group comprising: immunoglobulins, antibodies, antibody fragments, peptides, and polypeptides. In other embodiments, the second targeting moiety is selected from the group comprising: immunoglobulins, antibodies, antibody fragments, peptides, and polypeptides.

In some embodiments, the first target marker is selected from the group comprising: DNA, RNA, proteins, protein complexes, phosphorylated proteins, glycosylated proteins, folded proteins, and denatured proteins. In some embodiments, the second target marker is selected from the group comprising: DNA, RNA, proteins, protein complexes, phosphorylated proteins, glycosylated proteins, folded proteins, and denatured proteins. In certain embodiments the first and second targeting moieties are simultaneously selected from the group comprising: immunoglobulins, antibodies, antibody fragments, peptides, and polypeptides.

In some embodiments, the first and second target markers are simultaneously selected from the group comprising: DNA, RNA, proteins, protein complexes, phosphorylated proteins, glycosylated proteins, folded proteins, and denatured proteins. In certain embodiments, the bispecific imaging agent is a monoclonal antibody that recognizes two markers simultaneously.

In some embodiments, markers recognized simultaneously by the first and second targeting moieties are: Anaplastic lymphoma kinase, Alpha-fetoprotein (AFP), Beta-2-microglobulin (B2M), Beta-human chorionic gonadotropin (Beta-hCG), BCR-ABL, BRAF, CA15-3/CA27.29, CA19-9, CA-125, Calcitonin, Carcinoembryonic antigen (CEA), CD20, Chromogranin A (CgA), Cytokeratin, EGFR, Estrogen receptor (ER), progesterone receptor (PR), Fibrin/fibrinogen, HE4, HER2, Immunoglobulins, KIT, KRAS , Lactate dehydrogenase, Matrix metalloproteinases (MMP), Nuclear matrix protein 22, Prostate-specific antigen (PSA), Receptor Tyrosine kinases, Thyroglobulin, Urokinase plasminogen activator (uPA), and plasminogen activator inhibitor (PAI-1). In other embodiments, the markers recognized by the polyspecific agent include proteins within the receptor tyrosine kinase family. In other embodiments, markers recognized by the polyspecifc imaging agent include members of the EGFR family, including, but not limited to: EGFR, HER2, HER3, and HERO. In one embodiment, the polyspecific imaging agent binds EGFR and HER3 simultaneously as well as independently. In some embodiments, the imaging agent is monoclonal antibody, MEHD7945A, that binds bispecifically to EGFR and HER3, conjugated to a zirconium radiolabel.

Embodiments of the present invention are drawn to antibody-based monitoring and quantification of receptor tyrosine kinase signaling via tumor-associated or intratumoral receptor tyrosine kinases and antibody-based targeting of intratumoral receptor tyrosine kinases for therapeutic applications. The inventions disclosed herein are facilitated in part by the surprising discovery that antibodies and antibody polypeptides polyspecific for pairs of receptor tyrosine kinases such as EGFR and HER3 selectively bind several receptor tyrosine kinases simultaneously, including and preferentially as disclosed for the first time herein, tumor-associated and intratumoral receptor tyrosine kinases. The abilities of poly-specific antibodies to bind receptor tyrosine kinases at tumors in situ enables numerous diagnostic and therapeutic applications. For example, labeled antibody that quantitatively binds tumor-associated receptor tyrosine kinases can directly monitor responses to anti-receptor tyrosine kinase therapy in situ rather than indirectly through serum levels of receptor tyrosine kinases. Successful treatments result in a reduction in receptor tyrosine kinases signaling (e.g., through pharmaceutical compounds that block activation of the receptor), which may be reflected in a detectable decrease in the signal of the labeled antibody. It will be apparent to those of skill in the art that embodiments of the invention are applicable to any therapy that blocks receptor tyrosine kinases or causes apoptosis (e.g., chemotherapy), as well as any treatment that directly or indirectly reduces receptor tyrosine kinases levels (i.e., siRNA).

Alternatively or additionally, methods disclosed herein possess the ability to diagnose and treat diffuse receptor tyrosine kinases diseases; i.e., solid tumors, cancer and neurodegenerative disorders. As discussed above, detection, monitoring and treatment of cancer and its metastasis has proven particularly difficult. Surgical excision of metastatic lymph nodes, for example, frequently fails to remove all of the cancerous cells that may be present, greatly increasing the chances of recurrence. Moreover, current methods of monitoring of bone metastases are wholly inadequate as they image nearby normal bone repair rather than directly imaging the tumor, meaning that most scans cannot distinguish between malignant and non-malignant disease (i.e., other diseases that result in bone remodeling).

Capabilities of receptor tyrosine kinases in the evaluation and treatment of cancer and neurodegenerative disorder are particularly evident when used in conjunction with molecular imaging. Molecular imaging in oncology is the noninvasive imaging of key molecules and molecular-based events that are fundamental to human tumor biology. It can provide previously unavailable information regarding detection, differential diagnosis, tumor biology indicating proper therapeutic course, staging, reoccurrence and response to therapy. Nuclear medicine techniques in particular lend themselves to molecular imaging. Via radioactivity detectors, biomolecular radiotracers may be used to detect the real-time biochemistry of tumors, cancerous cells, and differentiated normal tissues, thereby providing qualitative or quantitative biochemical or functional information about human tumors and tissues.

Advances in molecular imaging have been facilitated through the development and improvement of monoclonal antibody technology. In addition to their recognized potential for cancer-targeted pharmaceuticals, monoclonal antibodies can be used as disease-specific contrast agents for diagnostic imaging. For example, by combining the high sensitivity and resolution of a positron emission tomography (“PET”) camera with the specificity of a monoclonal antibody, immuno-PET, the combination of PET with monoclonal antibodies, is an attractive, novel option to improve diagnostic tumor characterization. It will be appreciated, however, that concepts disclosed herein are not based on any single imaging technology. In fact, it is a technology adaptable to the use of almost any imaging parameter to infer qualitative or quantitative biochemical or functional information about human cells, tumors, neurons and tissues. Molecular imaging methods encompassed by the present disclosure include gamma camera imaging, single-photon emission computed tomography, positron emission tomography, magnetic resonance spectroscopy, magnetic resonance imaging, optical imaging, and ultrasound.

Immuno-PET is equivalent to comprehensive immunohistochemical staining in vivo, for which purpose the monoclonal antibody must be labeled with a positron emitter to enable visualization with a PET camera. However, there remains a need for the development of a new generation of monoclonal antibody-based imaging probes or novel radiotracers in addition to existing PET tracers, of which the non-tumor-specific metabolic tracer 18-fluoro-2-deoxy-D-glucose (¹⁸FDG) is currently used in >90% of all PET imaging procedures.

Deliberately engineering radiotracers to achieve success in tumors has proven challenging, resulting in the high attrition rate of novel radiotracers in the clinic. The target of a radiotracer necessarily frames its potential context of use (i.e. detection, response indicator), and candidates are often selected on the basis of preclinical evidence pointing to an upregulation in cancer. In this regard, it can be challenging to appropriately evaluate novel radiotracers in patients without a thorough appreciation of the patho-biological mechanism of target upregulation.

A particular embodiment of this invention has achieved success in this regard by demonstrating that changes in receptor tyrosine kinase-specific, EGFR and HERS-regulated gene expression and resulting translational products can be measured non-invasively with novel radiotracers derived from appropriately labeled antibodies specific to tumor-associated receptor tyrosine kinases. Radiotracers disclosed herein are highly specific for cancer cells in an receptor tyrosine kinase-dependent manner and can detect receptor tyrosine kinase-regulated elevations in receptor tyrosine kinases expression, demonstrating their ability to reflect intratumoral receptor tyrosine kinase signaling. In conjunction with imaging technologies known to those of skill in the art, the novel radiotracers disclosed herein allow for in situ quantification of receptor tyrosine kinase signaling via changes in receptor tyrosine kinases synthesis in response to pharmacological inhibition of the receptor tyrosine kinase. Moreover, the radiotracers possess unique polyspecificity multiple receptor tyrosine kinases simultaneously.

The ability of embodiments of the invention to monitor and image receptor tyrosine kinase signaling is applicable in a wide range of diagnostic methods. Embodiments of the invention can provide diagnostic information, and prognostic and predictive information related to cancer and metastasis of cancer cells. As mentioned previously, the ability to monitor tumor-associated receptor tyrosine kinase signaling facilitates extremely accurate measurements of therapeutic efficacy, particularly when anti-receptor tyrosine kinase treatments are used. In this capacity, embodiments of the invention can indicate when cancerous cells have developed adaptive mutations that render the cells unresponsive to receptor tyrosine kinase signaling. Furthermore, embodiments of the invention can be used to assess the stage of cancer and/or to provide information on the likely outcome of treatment. Embodiments of the invention can also predict whether a given course of therapy will be successful. For example, an elevated level of receptor tyrosine kinase signaling relative to a baseline reference may suggest a particularly aggressive course of anti-receptor tyrosine kinase treatment.

It will also be appreciated that the tumor-associated and intratumoral specificity of the anti-receptor tyrosine kinase polyspecific antibodies and antibody polypeptides disclosed herein permit targeting of anti-receptor tyrosine kinase and anti-cancer treatments to cells that express and/or display receptor tyrosine kinases. Tumor-associated receptor tyrosine kinases antibodies may be coupled to radioactive elements, cytotoxic nucleic acid analogues, apoptosis-inducing agents, and potentially any therapy now known or later developed that could benefit from accurate delivery to a specific population of cells. By binding solely to one or multiple receptor tyrosine kinases, the receptor tyrosine kinase antibodies and antibody polypeptides disclosed herein have the ability to target therapies directly to receptor tyrosine kinase-specific tumors, cancer cells, and neurons.

Embodiments of the present invention and methods disclosed herein can include any antibody now known or later discovered that is capable of specific binding to receptor tyrosine kinases, particularly tumor-associated receptor tyrosine kinases. As described above, the present invention also encompasses antibody fragments and characteristic portions of antibodies capable of specific binding to receptor tyrosine kinases. Select antibodies and antibody fragments may be used individually or in combination. When used in combination, the select antibodies and antibody fragments may be used simultaneously or sequentially. Antibodies and antibody fragments of the invention demonstrate peak tumor association between approximately about 72-120 hours post-administration, and in a particular embodiments within 72 hours post-administration.

Some embodiments of the invention utilize a monoclonal or polyclonal antibody (or characteristic fragment thererof) capable of specifically binding to a receptor tyrosine kinase epitope. Antibodies for use in embodiments of the invention may be from any species, e.g., human, mouse, rabbit, etc.

### Humanized and Veneered Antibodies

Monoclonal antibodies for use in embodiments of the invention may be developed by conventional means well known to those of skill in the art; hybridoma technology is but one example. See, e.g., G. Kohler and C. Milstein, Nature, 1975, 256: 495-497. Protocols for production of monoclonal antibodies and the cell lines that produce them are well known in the art. See, e.g., Gerhard et al, Proc. Natl. Acad. Sci. USA, 1978, 75:1510; Monoclonal Antibodies (R. Kennett, T. McKearn, & K. Bechtol eds. 1980); Monoclonal Antibodies and T-Cell Hybridomas (G. Hammerling, U. Hammerling, & J. Kearney eds. 1981); Kozbor et al, Proc. Natl. Acad. Sci. USA, 1982, 79:6651; Jonak et al, Hybridoma, 1983, 2:124; Monoclonal Antibodies and Functional Cell Lines (R. Kennett, K. Bechtol, & T. McKearn eds. 1983); and Shulman et al, Nature, 1982, 276:269-270.

In certain embodiments, particularly when using an anti-receptor tyrosine kinases antibody for therapeutic purposes, a humanized or veneered antibody may be used to reduce any potential immunogenic reaction. In general, humanized or veneered antibodies minimize unwanted immunological responses that limit the duration and effectiveness of therapeutic applications of non-human antibodies in human recipients.

A number of methods for preparing humanized antibodies comprising an antigen-binding portion derived from a non-human antibody have been described in the art. In particular, antibodies with rodent variable regions and their associated complementarity-determining regions (CDRs) fused to human constant domains have been described (e.g., see Winter et al., Nature, 1991, 349:293; Lobuglio et al., Proc. Nat. Acad. Sci. USA, 1989, 86:4220; Shaw et al., J. Immunol., 1987, 138:4534; and Brown et al., Cancer Res., 1987, 47:3577). Rodent CDRs grafted into a human supporting framework region (FR) prior to fusion with an appropriate human antibody constant domain (e.g., see Riechmann et al., Nature, 1988, 332:323; Verhoeyen et al., Science, 1988, 239:1534; and Jones et al. i Nature, 1986, 321:522) and rodent CDRs supported by recombinantly veneered rodent FRs have also been described (e.g., see EPO Patent Pub. No. 519,596).

Completely human antibodies are particularly desirable for therapeutic treatment of human patients. Such antibodies can be produced using transgenic mice that are incapable of expressing endogenous immunoglobulin heavy and light chains genes, but which can express human heavy and light chain genes (e.g., see Lonberg and Huszar, Int. Rev. Immunol., 1995, 13:65-93 and U.S. Pat. Nos. 5,545,806; 5,569,825; 5,625,126; 5,633,425; and 5,661,016).

Completely human antibodies or antigen-binding fragments thereof can also be identified and isolated from human antibody libraries. For example, expression products of a polynucleotide library encompassing the theoretical diversity of human antibodies (10^{12th} different antibodies), or physically realizable subportion thereof, can be screened with the totality and/or antigenic portions of the catalytic cleft to identify novel interacting antibodies or fragments. Exemplary libraries include phage-display libraries from immunized individuals (see, e.g., Barbas et al., J. Mol. Biol., 1993, 230:812-823), libraries of germline sequences (Griffiths et al., EMBO J., 1994, 13: 3245-3260) or naïve B-cell repertoires (Vaughan et al., Nature Biotech., 1996, 14:309-314). In particular embodiments, a library derived from a donor suffering from cancer may be used. In such libraries, receptor tyrosine kinase antigen stimulation increases mRNA production in B cells, which contributes to the isolation of heavy and light variable genes that are predisposed to receptor tyrosine kinase binding.

Synthetic libraries, in which germline antibody gene segments (VH, DH, and JH or Vκ/λ and Jκ/λ) are cloned and arranged combinatorially in vitro so as to reconstitute genes encoding complete VH and VL chains (see, e.g., Winter, FEBS Letters, 1998, 430:92-94), may also be used. See, e.g., de Kruif et al., J. Mol. Biol., 1995, 248: 97-105; Griffiths et al., EMBO J., 1994, 13: 3245-3260; Hoogenboom and Winter, J. Mol. Biol., 1992, 227:381-388; and Nissim et al., EMBO J., 1994, 13:692-698. Semi-synthetic libraries, which are generated by selecting one or more antibody frameworks as a scaffold and randomizing sequences within the CDR loops, may also be used. Particlar libraries may have fully or partially randomized CDR3 hypervariable regions of the heavy and/or light chains (see, e.g., Huls et al., Nat. Biotech., 1999, 17:276-281; Knappik et al. (J. Mol. Biol., 2000, 296: 57-86). See generally, Fuh, G. Expert. Opin. Biol. Ther., 2007, 7(1): 73-87; Kim et al., Mol. Cells, 2005, 20(1): 17-29. Phage, yeast, E. coli and ribosome display technologies may be used for library screening.

Veneered versions of anti-receptor tyrosine kinases antibodies may also be used in the methods of the present invention. The process of veneering involves selectively replacing FR residues from, e.g., a murine heavy or light chain variable region, with human FR residues in order to provide an antibody that comprises an antigen-binding portion that retains substantially all of the native FR protein folding structure. Veneering techniques are based on the understanding that antigen-binding characteristics are determined primarily by the structure and relative disposition of the heavy and light chain CDR sets within the antigen-association surface (e.g., see Davies et al., Ann. Rev. Biochem., 1990, 59:439). Thus, antigen association specificity can be preserved in a humanized antibody only wherein the CDR structures, their interaction with each other and their interaction with the rest of the variable region domains are carefully maintained. By using veneering techniques, exterior (e.g., solvent-accessible) FR residues that are readily encountered by the immune system are selectively replaced with human residues to provide a hybrid molecule that comprises either a weakly immunogenic, or substantially non-immunogenic veneered surface.

### Diagnostic Applications

In some embodiments, provided antibodies are used for diagnostic applications. Diagnostic assays may be employed to monitor the effects of various therapies upon receptor tyrosine kinase signaling in situ at the actual location of the tumor, as opposed to indirectly through the serum. Treatments that block or reduce receptor tyrosine kinase levels or receptor tyrosine kinase activation are reflected in a corresponding decrease in receptor tyrosine kinase regulated gene products such as receptor tyrosine kinases. Some embodiments of the invention monitor this effect in situ.

Diagnostic applications may also be able to detect the presence or absence of metastatic cancer disease, for example in the liver, lymph nodes or bone. In general the receptor tyrosine kinase-polyspecific antibodies provided herein may be administered before, during, or after any cancer-related treatment (e.g., anti-receptor tyrosine kinase treatments) to assess the effects of the treatment upon receptor tyrosine kinase signaling relative to a subject-specific baseline or a baseline derived from a population of patients. In particular embodiments, a population-derived baseline may comprise the mean or median of receptor tyrosine kinase signaling levels of receptor tyrosine kinase expression or activity in a group of patient or subjects that is not suffering from cancer.

In certain embodiments, binding can be detected by adding a detection entity to a provided antibody as discussed in the following section. In certain embodiments, the detection techniques of the present invention will include a negative control, which can involve applying the test to a control sample (e.g., from a normal non-cancerous tissue) so that the signal obtained thereby can be compared with the signal obtained from the sample being tested.

Particular diagnostic techniques for use in embodiments of the invention include, but are not limited to, enzyme linked immunosorbent assays (“ELISA”), positron emission tomography, Western blotting, immunohistochemistry, and magnetic resonance imaging.

Detection entities

In some embodiments, anti-receptor tyrosine kinase polyspecific antibodies are used for and with detection applications. Multifunctional agents described herein may be used which comprise at least one detection entity, in addition to a provided antibody as described herein.

A detection entity may be any entity that allows detection of receptor tyrosine kinases antibody or antibody polypeptides after binding to a receptor tyrosine kinases in a tissue of interest; e.g., cancer cells or bone. Any of a wide variety of detectable agents can be used as detection entity (e.g., labeling moieties) in multifunctional antibody agents of the provided antibodies. A detection entity may be directly detectable or indirectly detectable. Examples of detection entities include, but are not limited to: various ligands, radionuclides (e.g., ³H,¹⁴C, ¹⁸F, ¹⁹F, ³²P, ³⁵S, ¹³⁵I, ¹²⁵I, ¹²³I, ⁶⁴Cu, ¹⁸⁷Re, ¹¹¹In, ⁹⁰Y, ^{99m}Tc, ¹⁷⁷Lu, ⁸⁹Zr, etc.), fluorescent dyes (for specific exemplary fluorescent dyes, see below), chemiluminescent agents (such as, for example, acridinum esters, stabilized dioxetanes, and the like), bioluminescent agents, spectrally resolvable inorganic fluorescent semiconductors nanocrystals (i.e., quantum dots), metal nanoparticles (e.g., gold, silver, copper, platinum, etc.) nanoclusters, paramagnetic metal ions, enzymes (for specific examples of enzymes, see below), colorimetric labels (such as, for example, dyes, colloidal gold, and the like), biotin, dioxigenin, haptens, and proteins for which antisera or monoclonal antibodies are available.

In certain embodiments, a detection entity comprises a fluorescent label. Numerous known fluorescent labeling moieties of a wide variety of chemical structures and physical characteristics are suitable for use in the present invention. Suitable fluorescent dyes include, but are not limited to, fluorescein and fluorescein dyes (e.g., fluorescein isothiocyanine or FITC, naphthofluorescein, 4′,5′-dichloro-2′,7′- dimethoxyfluorescein, (3 carboxyfluorescein or FAM, etc.), carbocyanine, merocyanine, styryl dyes, oxonol dyes, phycoerythrin, erythrosin, eosin, rhodamine dyes (e.g., carboxytetramethyl- rhodamine or TAMRA, carboxyrhodamine 6G, carboxy-X-rhodamine (ROX), lissamine rhodamine B, rhodamine 6G, rhodamine Green, rhodamine Red, tetramethylrhodamine (TMR), etc.), coumarin and coumarin dyes (e.g. , methoxycoumarin, dialkylaminocoumarin, hydroxycoumarin, aminomethylcoumarin (AMCA), etc.), Oregon Green Dyes (e.g., Oregon Green 488, Oregon Green 500, Oregon Green 514., etc.), Texas Red, Texas Red-X, Spectrum Red™, Spectrum Green™, cyanine dyes (e.g., Cy-3™, Cy-5™, Cy-3.5™, Cy-5.5™ etc.), Alexa Fluor dyes (e.g., Alexa Fluor 350, Alexa Fluor 488, Alexa Fluor 532, Alexa Fluor 546, Alexa Fluor 568, Alexa Fluor 594, Alexa Fluor 633, Alexa Fluor 660, Alexa Fluor 680, etc.), BODIPY dyes (e.g., BODIPY FL, BODIPY R6G, BODIPY TMR, BODIPY TR, BODIPY 530/550, BODIPY 558/568, BODIPY 564/570, BODIPY 576/5⁸⁹, BODIPY 581/591, BODIPY 630/650, BODIPY 650/665, etc.), IRDyes (e.g., IRD40, IRD 700, IRD 800, etc.), and the like. For more examples of suitable fluorescent dyes and methods for coupling fluorescent dyes to other chemical entities such as proteins and peptides, see, for example, “The Handbook of Fluorescent Probes and Research Products”, 9111 Ed., Molecular Probes, Inc., Eugene, Oreg.

Favorable properties of fluorescent labeling agents include high molar absorption coefficient, high fluorescence quantum yield, and photostability. In certain embodiments, labeling fluorophores desirably exhibit absorption and emission wavelengths in the visible (i.e., between 400 and 750 nm) rather than in the ultraviolet range of the spectrum (i.e., lower than 400 nm).

In certain embodiments, a detection entity comprises an enzyme. Examples of suitable enzymes include, but are not limited to, those used in an ELISA, e.g., horseradish peroxidase, beta-galactosidase, luciferase, alkaline phosphatase, etc. Other examples include beta-glucuronidase, beta-D-glucosidase, urease, glucose oxidase, etc. An enzyme may be conjugated to a targeting entity (e.g., chlorotoxin moiety) using a linker group such as a carbodiimide, a diisocyanate, a glutaraldehyde, and the like. More detailed description of suitable linkers is provided elsewhere herein.

In certain embodiments, a detection entity comprises a radioisotope that is detectable by Single Photon Emission Computed Tomography (SPECT) or Position Emission Tomography (PET). The high resolution and quantitative imaging of PET is particularly suited to certain embodiments of the invention. Examples of such radionuclides include, but are not limited to, zirconium-89 (⁸⁹Zr), iodine-124 (¹²⁴I) iodine-131 (¹³¹I), iodine-125 (¹²⁵I), bismuth-212 (²¹²Bi), bismuth-213 (²¹³Bi), astatine-221 (²¹¹At), copper-67 (⁶⁷Cu), copper-64 (⁶⁴Cu), rhenium-186 (¹⁸⁶_{Re}), rhenium-186 (¹⁸⁸Re), phosphorus-32 (³²P), samarium- 153 (¹⁵³Sm), lutetium-177 (¹¹⁷Lu), technetium-99m (^{99m}Tc), gallium-67 (⁶⁷Ga), indium-111 (¹¹¹In), and thallium-201 (²⁰¹Tl) . Particular procedures for the production and use of ⁸⁹Zr-labeled monoclonal antibodies are disclosed in Verel, I. et al. “⁸⁹Zr-Immuno-PET: Comprehensive Procedures for the Production of⁸⁹Zr-Labeled Monoclonal Antibodies”, J. Nucl. Med. 2003; 44:1271-1281, incorporated by reference herein.

In certain embodiments, a labeling moiety comprises a radioisotope that is detectable by Gamma camera. Examples of such radioisotopes include, but are not limited to, iodine-131 (¹³¹I), and technetium-99m (^{99m}Tc).

In certain embodiments chelators or bonding moieties for diagnostic and therapeutic radiopharmaceuticals are also contemplated and can be chemically associated with the imaging agents. Exemplary chelators can be selected to form stable complexes with radioisotopes that have imageable gamma ray or positron emissions, such as ^{99m}Tc, ¹¹¹In, ⁶⁴Cu, and ⁶⁷Ga. Exemplary chelators include diaminedithiols, monoamine-monoamidedithiols, triamide-monothiols, monoamine-diamide-monothiols, diaminedioximes, and hydrazines. Chelators generally are tetradentate with donor atoms selected from nitrogen, oxygen and sulfur, and may include for example, cyclic and acyclic polyaminocarboxylates such as diethylenetriaminepentaacetic acid (DTPA), 1,4,7,10-tetraazacyclododecane-1,4,7,10-tetraacetic acid (DOTA), (DO3A), 2-benzyl-DOTA, alpha-(2-phenethyl)1,4,7,10-tetraazazcyclododecane-1-acetic-4,7,10-tris(methylacetic)acid, 2-benzyl-cyclohexyldiethylenetriaminepentaacetic acid, 2-benzyl-6-methyl-DTPA, and 6,6″-bis[N,N,N″,N″-tetra(carboxymethyl)aminomethyl)-4′-(3-amino-4-methoxyphenyl)-2,2′:6′,2″-terpyridine.

In certain embodiments, a detection entity comprises a paramagnetic metal ion that is a good contrast enhancer in Magnetic Resonance Imaging (MRI). Examples of such paramagnetic metal ions include, but are not limited to, gadolinium III (Gd3+), chromium III (Cr3+), dysprosium III (Dy3+), iron III (Fe3+), manganese II (Mn2+), and ytterbium III (Yb3+). In certain embodiments, the detection entity comprises gadolinium III (Gd3+). Gadolinium is an FDA-approved contrast agent for MRI, which accumulates in abnormal tissues causing these abnormal areas to become very bright (enhanced) on the magnetic resonance image. Gadolinium is known to provide great contrast between normal and abnormal tissues in different areas of the body, in particular in the brain.

In certain embodiments, a labeling moiety comprises a stable paramagnetic isotope detectable by nuclear magnetic resonance spectroscopy (MRS). Examples of suitable stable paramagnetic isotopes include, but are not limited to, carbon-13 (¹³C) and fluorine-19 (¹⁹F).

### Pharmaceutical Compositions

The present invention also provides compositions comprising one or more provided antibodies, fragments or characteristic portions thereof. In some embodiments, the present invention provides at least one antibody and at least one pharmaceutically acceptable excipient. Such pharmaceutical compositions may optionally comprise and/or be administered in combination with one or more additional therapeutically or biologically active substances. In some embodiments, provided pharmaceutical compositions are useful in medicine or the manufacture of medicaments. In some embodiments, provided pharmaceutical compositions are useful as prophylactic agents (i.e., vaccines) in the treatment or prevention of cancer and neurodegenerative disorders thereof. In some embodiments, provided pharmaceutical compositions are useful in therapeutic applications, for example in individuals suffering from cancer; e.g., as delivery vehicles capable of specifically targeting cytotoxic agents or compounds that block receptor tyrosine kinase signaling. In some embodiments, the pharmaceutical compositions are simultaneously useful in diagnostic applications and therapeutic applications. In some embodiments, pharmaceutical compositions are formulated for administration to humans. In some embodiments, the pharmaceutical compositions comprise an anti-receptor tyrosine kinases antibody in combination with or conjugated to an anti-cancer agent or other therapeutic as defined herein.

For example, pharmaceutical compositions may be provided in a sterile injectable form (e.g., a form that is suitable for subcutaneous injection or intravenous infusion). In some embodiments, pharmaceutical compositions are provided in a liquid dosage form that is suitable for injection. In some embodiments, pharmaceutical compositions are provided as powders (e.g., lyophilized and/or sterilized), optionally under vacuum, which are reconstituted with an aqueous diluent (e.g., water, buffer, salt solution, etc.) prior to injection. In some embodiments, pharmaceutical compositions are diluted and/or reconstituted in water, sodium chloride solution, sodium acetate solution, benzyl alcohol solution, phosphate buffered saline, etc. In some embodiments, powder should be mixed gently with the aqueous diluent (e.g., not shaken).

In some embodiments, provided pharmaceutical compositions comprise one or more pharmaceutically acceptable excipients (e.g., preservative, inert diluent, dispersing agent, surface active agent and/or emulsifier, buffering agent, etc.). In some embodiments, pharmaceutical compositions comprise one or more preservatives. In some embodiments, pharmaceutical compositions comprise no preservatives.

In some embodiments, pharmaceutical compositions are provided in a form that can be refrigerated and/or frozen. In some embodiments, pharmaceutical compositions are provided in a form that cannot be refrigerated and/or frozen. In some embodiments, reconstituted solutions and/or liquid dosage forms may be stored for a certain period of time after reconstitution (e.g., 2 hours, 12 hours, 24 hours, 2 days, 5 days, 7 days, 10 days, 2 weeks, a month, two months, or longer). In some embodiments, storage of antibody compositions for longer than the specified time results in antibody degradation.

Liquid dosage forms and/or reconstituted solutions may comprise particulate matter and/or discoloration prior to administration. In some embodiments, a solution should not be used if discolored or cloudy and/or if particulate matter remains after filtration.

Pharmaceutical compositions described herein may be prepared by any method known or hereafter developed in the art of pharmacology. In some embodiments, such preparatory methods include the step of bringing active ingredient into association with one or more excipients and/or one or more other accessory ingredients, and then, if necessary and/or desirable, shaping and/or packaging the product into a desired single- or multi-dose unit.

A pharmaceutical composition in accordance with the invention may be prepared, packaged, and/or sold in bulk, as a single unit dose, and/or as a plurality of single unit doses. As used herein, a “unit dose” is discrete amount of the pharmaceutical composition comprising a predetermined amount of the active ingredient; for example, an anti-receptor tyrosine kinases antibody and an anti-receptor tyrosine kinase therapy. The amount of the active ingredient is generally equal to a dose that would be administered to a subject and/or a convenient fraction of such a dose such as, for example, one-half or one-third of such a dose.

Relative amounts of active ingredient, pharmaceutically acceptable excipient, and/or any additional ingredients in a pharmaceutical composition in accordance with the invention may vary, depending upon the identity, size, and/or condition of the subject treated and/or depending upon the route by which the composition is to be administered. By way of example, the composition may comprise between 0.1% and 100% (w/w) active ingredient.

Pharmaceutical compositions of the present invention may additionally comprise a pharmaceutically acceptable excipient, which, as used herein, may be or comprise solvents, dispersion media, diluents, or other liquid vehicles, dispersion or suspension aids, surface active agents, isotonic agents, thickening or emulsifying agents, preservatives, solid binders, lubricants and the like, as suited to the particular dosage form desired. Remington's The Science and Practice of Pharmacy, 21st Edition, A. R. Gennaro, (Lippincott, Williams & Wilkins, Baltimore, MD, 2006) discloses various excipients used in formulating pharmaceutical compositions and known techniques for the preparation thereof. Except insofar as any conventional excipient medium is incompatible with a substance or its derivatives, such as by producing any undesirable biological effect or otherwise interacting in a deleterious manner with any other component(s) of the pharmaceutical composition, its use is contemplated to be within the scope of this invention.

### Conjugates Generally

Multifunctional agents described herein comprise multiple entities, each having at least one function (e.g., a monoclonal antibody specific for an epitope in or near the site of two receptor tyrosine kinases conjugated to a chemotherapeutic agent). Certain embodiments of contemplated multifunctional agents comprise a targeting entity and at least one of the following entities: a detection entity, a therapeutic entity, and a diagnostic entity. In some embodiments, a multifunctional agent comprising an anti-receptor tyrosine kinases antibody contains a targeting entity and a detection entity but not a therapeutic entity. In some embodiments, a multifunctional agent of the invention contains a targeting entity, a therapeutic entity and a detection entity. In some embodiments, the entities of an agent may be conjugated to one another. Conjugation of various entities to form a multifunctional agent is not limited to particular modes of conjugation. For example, two entities may be covalently conjugated directly to each other. Alternatively, two entities may be indirectly conjugated to each other, such as via a linker entity. In some embodiments, a multifunctional agent may include different types of conjugation within the agent, such that some entities of the agent are conjugated via direct conjugation while other entities of the agent are indirectly conjugated via one or more linkers. In some embodiments, a multifunctional agent of the invention comprises a single type of a linker entity. In other embodiments, a multifunctional agent of the invention comprises more than one type of linker entities. In some embodiments, a multifunctional agent includes a single type of linker entities but of varying length.

In some embodiments, there is a covalent association between or among entities contained in a multifunctional agent. As will be appreciated by one skilled in the art, the moieties may be attached to each other either directly or indirectly (e.g., through a linker, as described below).

In some embodiments, where one entity (such as a targeting entity) and a second entity of a multifunctional agent are directly covalently linked to each other, such direct covalent conjugation can be through a linkage (e.g., a linker or linking entity) such as an amide, ester, carbon-carbon, disulfide, carbamate, ether, thioether, urea, thiourea, isothiourea, amine, or carbonate linkage. Covalent conjugation can be achieved by taking advantage of functional groups present on the first entity and/or the second entity of the multifunctional agent. Alternatively, a non-critical amino acid may be replaced by another amino acid that will introduce a useful group (such as amino, carboxy or sulfhydryl) for coupling purposes. Alternatively, an additional amino acid may be added to at least one of the entities of the multifunctional agent to introduce a useful group (such as amino, carboxy or sulfhydryl) for coupling purposes. Suitable functional groups that can be used to attach moieties together include, but are not limited to, amines, anhydrides, hydroxyl groups, carboxy groups, thiols, and the like. An activating agent, such as a carbodiimide, can be used to form a direct linkage. A wide variety of activating agents are known in the art and are suitable for conjugating one entity to a second entity.

In other embodiments, entities of a multifunctional agent embraced by the present invention are indirectly covalently linked to each other via a linker group. Such a linker group may also be referred to as a linker or a linking entity. This can be accomplished by using any number of stable bifunctional agents well known in the art, including homofunctional and heterofunctional agents (for examples of such agents, see, e.g., Pierce Catalog and Handbook). The use of a bifunctional linker differs from the use of an activating agent in that the former results in a linking moiety being present in the resulting conjugate (agent), whereas the latter results in a direct coupling between the two moieties involved in the reaction. The role of a bifunctional linker may be to allow reaction between two otherwise inert moieties. Alternatively or additionally, the bifunctional linker that becomes part of the reaction product may be selected such that it confers some degree of conformational flexibility to the anti-receptor tyrosine kinases antibody (e.g., the bifunctional linker comprises a straight alkyl chain containing several atoms, for example, the straight alkyl chain contains between 2 and 10 carbon atoms). Alternatively or additionally, the bifunctional linker may be selected such that the linkage formed between a provided antibody and therapeutic agent is cleavable, e.g., hydrolysable (for examples of such linkers, see e.g. U.S. Pat. Nos. 5,773,001; 5,739,116 and 5,877,296, each of which is incorporated herein by reference in its entirety). Such linkers, for example, may be used when higher activity of certain entities, such as a targeting agent (e.g., the provided receptor tyrosine kinases-specific antibodies) and/or of a therapeutic entity is observed after hydrolysis of the conjugate. Exemplary mechanisms by which an entity may be cleaved from a multifunctional agent include hydrolysis in the acidic pH of the lysosomes (hydrazones, acetals, and cis-aconitate-like amides), peptide cleavage by lysosomal enzymes (the capthepsins and other lysosomal enzymes), and reduction of disulfides). Another mechanism by which such an entity is cleaved from the multifunctional agent includes hydrolysis at physiological pH extra- or intra-cellularly. This mechanism applies when the crosslinker used to couple one entity to another entity is a biodegradable/bioerodible component, such as polydextran and the like.

For example, hydrazone-containing multifunctional agents can be made with introduced carbonyl groups that provide the desired release properties. Multifunctional agents can also be made with a linker that comprises an alkyl chain with a disulfide group at one end and a hydrazine derivative at the other end. Linkers containing functional groups other than hydrazones also have the potential to be cleaved in the acidic milieu of lysosomes. For example, multifunctional agents can be made from thiol-reactive linkers that contain a group other than a hydrazone that is cleavable intracellularly, such as esters, amides, and acetals/ketals.

Another example of class of pH sensitive linkers are the cis-aconitates, which have a carboxylic acid group juxtaposed to an amide group. The carboxylic acid accelerates amide hydrolysis in the acidic lysosomes. Linkers that achieve a similar type of hydrolysis rate acceleration with several other types of structures can also be used.

Another potential release method for conjugates of the anti-receptor tyrosine kinases antibodies is the enzymatic hydrolysis of peptides by the lysosomal enzymes. In one example, a provided antibody is attached via an amide bond to para-aminobenzyl alcohol and then a carbamate or carbonate is made between the benzyl alcohol and the therapeutic agent. Cleavage of the peptide leads to collapse of the amino benzyl carbamate or carbonate, and release of the therapeutic agent. In another example, a phenol can be cleaved by collapse of the linker instead of the carbamate. In another variation, disulfide reduction is used to initiate the collapse of a para-mercaptobenzyl carbamate or carbonate.

Useful linkers which may be used as a linking entity of a multifunctional agent provided herein include, without limitation: polyethylene glycol, a copolymer of ethylene glycol, a polypropylene glycol, a copolymer of propylene glycol, a carboxymethylcellulose, a polyvinyl pyrrolidone, a poly-1,3-dioxolane, a poly-1,3,6-trioxane, an ethylene/maleic anhydride copolymer, a polyaminoacid, a dextran n-vinyl pyrrolidone, a poly n-vinyl pyrrolidone, a propylene glycol homopolymer, a propylene oxide polymer, an ethylene oxide polymer, a polyoxyethylated polyol, a polyvinyl alcohol, a linear or branched glycosylated chain, a polyacetal, a long chain fatty acid, a long chain hydrophobic aliphatic group.

Some embodiments of the invention utilize multifunctional agents that include at least one non-covalently associated entity. Examples of non-covalent interactions include, but are not limited to, hydrophobic interactions, electrostatic interactions, dipole interactions, van der Waals interactions, and hydrogen bonding. Irrespective of the nature of the binding, interaction, or coupling, the association between a first entity and a second entity is, in some embodiments, selective, specific and strong enough so that the second entity contained in the agent does not dissociate from the first entity before or during transport/delivery to and into the target. Thus, association among multiple entities of a multifunctional agent may be achieved using any chemical, biochemical, enzymatic, or genetic coupling known to one skilled in the art.

### Therapeutic Conjugates

As described herein, anti-receptor tyrosine kinases antibodies may comprise part of multifunctional agents with therapeutic utility related to cancer or neurodegenerative disorders. Examples of therapeutic utilities in the context of the present disclosure include, without limitation, utility associated with targeting (e.g., receptor tyrosine kinases-specific monoclonal antibody), utility associated with therapeutic effects (e.g., cytotoxic and/or cytostatic effects, anti-proliferative effects, anti-angiogenic effects, reducing symptoms etc.), and utility associated with diagnosis, detection or labeling, etc.

A targeting entity is a molecular structure that can be contained in an agent which affects or controls the site of action by specifically interacting with, or has affinity for, a target of interest. As an example, a target may be a molecule or molecular complex present on a cell surface, e.g., certain cell types, tissues, etc. In some embodiments of the invention, the target is tumor-associated or intratumoral receptor tyrosine kinases and the targeting entity is an anti-receptor tyrosine kinases antibody. The anti-receptor tyrosine kinase targeting entities disclosed herein can, by virtue of their affinity for epitopes that are only available in uncomplexed receptor tyrosine kinase, specifically or preferentially interact with receptor tyrosine kinases. Use of targeting moieties for agents such as therapeutic agents is known in the art. In the context of the present application, primary or metastatic cancer cells as well as neuronal cells are the target. That is, at the molecular level, a target is a molecule or cellular constituent that is present (e.g., preferentially expressed) on a cancer cell or neuronal cell, such that it can specifically or preferentially bind to an anti-receptor tyrosine kinases antibody upon contact. The anti-receptor tyrosine kinases antibodies of the invention exert specificity for their target (e.g., receptor tyrosine kinases of cancer and neuronal cells) and are able to localize to and bind to the target. In some embodiments, anti-receptor tyrosine kinases antibody targeting entities localize to cancer cells and neurons and retain their association over a period of time. In some embodiments, the receptor tyrosine kinase targets are intratumoral and/or integral membrane proteins.

In some embodiments, the anti-receptor tyrosine kinases antibodies are multifunctional agents comprising a receptor tyrosine kinases targeting entity, which essentially consists of a receptor tyrosine kinases-specific antibodies or antigen-binding fragment thereof, conjugated to one or more therapeutic agents. In such embodiments, therefore, the multifunctional agents are antibody conjugates. Non-limiting embodiments of useful conjugates of anti-receptor tyrosine kinases antibodies that may be used in the diagnosis or assessment of, treatment of and the manufacture of medicaments for cancer or neurodegenerative disorders are provided below.

In some embodiments, anti-receptor tyrosine kinases antibodies are conjugated to a nucleic acid molecule that is useful as a therapeutic agent for treating cancer or a neurodegenerative disorder. A variety of chemical types and structural forms of nucleic acid can be suitable for such strategies. These include, by way of non-limiting example, DNA, including single-stranded (ssDNA) and double-stranded (dsDNA); RNA, including, but not limited to ssRNA, dsRNA, tRNA, mRNA, rRNA, enzymatic RNA; RNA:DNA hybrids, triplexed DNA (e.g., dsDNA in association with a short oligonucleotide), and the like.

In some embodiments, the nucleic acid agent is between about 5 and 2000 nucleotides long. In some embodiments, the nucleic acid agent is at least about 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more nucleotides long. In some embodiments, the nucleic acid agent is less than about 2000, 1900, 1800, 1700, 1600, 1500, 1400, 1300, 1200, 1100, 1000, 900, 800, 700, 600, 500, 450, 400, 350, 300, 250, 200, 150, 100, 50, 45, 40, 35, 30, 25, 20 or fewer nucleotides long.

In some embodiments, the nucleic acid agent comprises a promoter and/or other sequences that regulate transcription. Such embodiments may comprise, for example, nucleotides sequences corresponding to response elements. In some embodiments, the nucleic acid agent comprises an origin of replication and/or other sequences that regulate replication. In some embodiments, the nucleic acid agent does not include a promoter and/or an origin of replication.

Nucleic acid anti-cancer agents suitable for use in the practice of the present invention include those agents that target genes associated with tumorigenesis and cell growth or cell transformation (e.g., proto-oncogenes, which code for proteins that stimulate cell division), angiogenic/anti-angiogenic genes, tumor suppressor genes (which code for proteins that suppress cell division), genes encoding proteins associated with tumor growth and/or tumor migration, and suicide genes (which induce apoptosis or other forms of cell death), especially suicide genes that are most active in rapidly dividing cells.

Examples of genes associated with tumorigenesis and/or cell transformation include receptor tyrosine kinase genes, MLL fusion genes, BCR-ABL, TEL-AML1, EWS-FLI1, TLS-FUS, PAX3-FKHR, Bc1-2, AML1-ETO, AML1-MTG8, Ras, Fos PDGF, RET, APC, NF-1, Rb, p53, MDM2 and the like; overexpressed genes such as multidrug resistance genes; cyclins; beta-Catenin; telomerase genes; c-myc, n-myc, Bc1-2, Erb-B1 and Erb-B2; and mutated genes such as Ras, Mos, Raf, and Met. Examples of tumor suppressor genes include, but are not limited to, p53, p21, RB1, WT1, NF1, VHL, APC, DAP kinase, p16, ARF, Neurofibromin, and PTEN. Examples of genes that can be targeted by nucleic acid agents useful in anti-cancer therapy include genes encoding proteins associated with tumor migration such as integrins, selectins, and metalloproteinases; anti-angiogenic genes encoding proteins that promote formation of new vessels such as Vascular Endothelial Growth Factor (VEGF) or VEGFr; anti-angiogenic genes encoding proteins that inhibit neovascularization such as endostatin, angiostatin, and VEGF-R2; and genes encoding proteins such as interleukins, interferon, fibroblast growth factor (α-FGF and (β-FGF), insulin-like growth factor (e.g., IGF-1 and IGF-2), Platelet-derived growth factor (PDGF), tumor necrosis factor (TNF), Transforming Growth Factor (e.g., TGF-α and TGF-β, Epidermal growth factor (EGF), Keratinocyte Growth Factor (KGF), stem cell factor and its receptor c-Kit (SCF/c-Kit) ligand, CD40L/CD40, VLA-4 VCAM-1, ICAM-1/LFA-1, hyalurin/CD44, and the like.

Nucleic acid agents suitable for conjugation with anti-receptor tyrosine kinases antibodies may have any of a variety of uses including, for example, use as anti-cancer or other therapeutic agents, probes, primers, etc. Nucleic acid agents may have enzymatic activity (e.g., ribozyme activity), gene expression inhibitory activity (e.g., as antisense or siRNA agents, etc), and/or other activities. Nucleic acids agents may be active themselves or may be vectors that deliver active nucleic acid agents (e.g., through replication and/or transcription of a delivered nucleic acid). For purposes of the present specification, such vector nucleic acids are considered “therapeutic agents” if they encode or otherwise deliver a therapeutically active agent, even if they do not themselves have therapeutic activity.

In certain embodiments, conjugates of anti-receptor tyrosine kinases antibodies comprise a nucleic acid therapeutic agent that comprises or encodes an antisense compound. The terms “antisense compound or agent,” “antisense oligomer,” “antisense oligonucleotide,” and “antisense oligonucleotide analog” are used herein interchangeably, and refer to a sequence of nucleotide bases and a subunit-to-subunit backbone that allows the antisense compound to hybridize to a target sequence in an RNA by Watson-Crick base pairing to form an RNA oligomer heteroduplex within the target sequence. The oligomer may have exact sequence complementarity within the target sequence or near complementarity. Such antisense oligomers may block or inhibit translation of the mRNA containing the target sequence, or inhibit gene transcription. Antisense oligomers may bind to double-stranded or single-stranded sequences.

Examples of antisense oligonucleotides suitable for use in the practice of the present invention include, for example, those mentioned in the following reviews: R. A Stahel et al., Lung Cancer, 2003, 41: S81-S88; K. F. Pirollo et al., Pharmacol. Ther., 2003, 99: 55-77; A. C. Stephens and R. P. Rivers, Curr. Opin. Mol. Ther., 2003, 5: 118-122; N. M. Dean and C. F. Bennett, Oncogene, 2003, 22: 9087-9096; N. Schiavone et al., Curr. Pharm. Des., 2004, 10: 769-784; L. Vidal et al., Eur. J. Cancer, 2005, 41: 2812-2818; T. Aboul-Fadl, Curr. Med. Chem., 2005, 12: 2193-2214; M. E. Gleave and B. P. Monia, Nat. Rev. Cancer, 2005, 5: 468-479; Y. S. Cho-Chung, Curr. Pharm. Des., 2005, 11: 2811-2823; E. Rayburn et al., Lett. Drug Design & Discov., 2005, 2: 1-18; E. R. Rayburn et al., Expert Opin. Emerg. Drugs, 2006, 11: 337-352; I. Tamm and M. Wagner, Mol. Biotechnol., 2006, 33: 221-238 (each of which is incorporated herein by reference in its entirety).

Examples of suitable antisense oligonucleotides include oblimersen sodium (also known as G31239, developed by Genta, Inc., Berkeley Heights, N.J.), a phosphorothioate oligomer targeted towards the initiation codon region of the bc1-2 mRNA. Bc1-2 is a potent inhibitor of apoptosis and is overexpressed in many cancers including follicular lymphomas, breast cancer, colon cancer, cancer, and intermediate/high-grade lymphomas (C. A. Stein et al., Semin. Oncol., 2005, 32: 563-573; S. R. Frankel, Semin. Oncol., 2003, 30: 300-304). Other suitable antisense oligonucleotides include GEM-231 (HYB0165, Hybridon, Inc., Cambridge, Mass.), which is a mixed backbone oligonucleotide directed against cAMP-dependent protein kinase A (PKA) (S. Goel et al., Clin. Cancer Res., 2003, 9: 4069-4076); Affinitak (ISIS 3521 or aprinocarsen, ISIS pharmaceuticals, Inc., Carlsbad, Calif.), an antisense inhibitor of PKCalpha; OGX-011 (Isis 112989, Isis Pharmaceuticals, Inc.), a 2′-methoxyethyl modified antisense oligonucleotide against clusterin, a glycoprotein implicated in the regulation of the cell cycle, tissue remodeling, lipid transport, and cell death and which is overexpressed in cancers of breast, prostate and colon; ISIS 5132 (Isis 112989, Isis Pharmaceuticals, Inc.), a phosphorothioate oligonucleotide complementary to a sequence of the 3′-unstranslated region of the c-raf-1 mRNA (S. P. Henry et al., Anticancer Drug Des., 1997, 12: 409-420; B. P. Monia et al., Proc. Natl. Acad. Sci. USA, 1996, 93: 15481- 15484; C. M. Rudin et al., Clin. Cancer Res., 2001, 7: 1214-1220); ISIS 2503 (Isis Pharmaceuticals, Inc.), a phosphorothioate oligonucleotide antisense inhibitor of human H-ras mRNA expression (J. Kurreck, Eur. J. Biochem., 2003, 270: 1628-1644); oligonucleotides targeting the X-linked inhibitor of apoptosis protein (XIAP), which blocks a substantial portion of the apoptosis pathway, such as GEM 640 (AEG 35156, Aegera Therapeutics Inc. and Hybridon, Inc.) or targeting survivin, an inhibitor of apoptosis protein (IAP), such as ISIS 23722 (Isis Pharmaceuticals, Inc.), a 2′-O-methoxyethyl chimeric oligonucleotide; MG98, which targets DNA methyl transferase; and GTI-2040 (Lorus Therapeutics, Inc. Toronto, Canada), a 20-mer oligonucleotide that is complementary to a coding region in the mRNA of the R2 small subunit component of human ribonucleotide reductase.

Other suitable antisense oligonucleotides include antisense oligonucleotides that are being developed against Her-2/neu, c-Myb, c-Myc, and c-Raf (see, for example, A. Biroccio et al., Oncogene, 2003, 22: 6579-6588; Y. Lee et al., Cancer Res., 2003, 63: 2802-2811; B. Lu et al., Cancer Res., 2004, 64: 2840-2845; K. F. Pirollo et al., Pharmacol. Ther., 2003, 99: 55-77; and A. Rait et al., Ann. N. Y. Acad. Sci., 2003, 1002: 78-89).

In certain embodiments, conjugates of anti-receptor tyrosine kinases antibodies comprise a nucleic acid therapeutic agent that comprises or encodes an interfering RNA molecule. The terms “interfering RNA” and “interfering RNA molecule” are used herein interchangeably, and refer to an RNA molecule that can inhibit or downregulate gene expression or silence a gene in a sequence-specific manner, for example by mediating RNA interference (RNAi). RNA interference (RNAi) is an evolutionarily conserved, sequence-specific mechanism triggered by double-stranded RNA (dsRNA) that induces degradation of complementary target single-stranded mRNA and “silencing” of the corresponding translated sequences (McManus and Sharp, 2002, Nature Rev. Genet., 2002, 3: 737). RNAi functions by enzymatic cleavage of longer dsRNA strands into biologically active “short-interfering RNA” (siRNA) sequences of about 21-23 nucleotides in length (Elbashir et al., Genes Dev., 2001, 15: 188). RNA interference has emerged as a promising approach for therapy of cancer and other disorders.

An interfering RNA suitable for use in the practice of the present invention can be provided in any of several forms. For example, an interfering RNA can be provided as one or more of an isolated short interfering RNA (siRNA), double-stranded RNA (dsRNA), micro-RNA (miRNA), or short hairpin RNA (shRNA).

Examples of interfering RNA molecules suitable for use in the present invention include, for example, the iRNAs cited in the following reviews: O. Milhavet et al., Pharmacol. Rev., 2003, 55: 629-648; F. Bi et al., Curr. Gene. Ther., 2003, 3: 411- 417; P. Y. Lu et al., Curr. Opin. Mol. Ther., 2003, 5: 225-234; I. Friedrich et al., Semin. Cancer Biol., 2004, 14: 223-230; M. Izquierdo, Cancer Gene Ther., 2005, 12: 217-227; P. Y. Lu et al., Adv. Genet., 2005, 54: 117-142; G. R. Devi, Cancer Gene Ther., 2006, 13: 819-829; M. A. Behlke, Mol. Ther., 2006, 13: 644-670; and L. N. Putral et al., Drug News Perspect., 2006, 19: 317-324 (the contents of each of which are incorporated herein by reference in their entirety).

Other examples of suitable interfering RNA molecules include, but are not limited to, p53 interfering RNAs (e.g., T. R. Brummelkamp et al., Science, 2002, 296: 550-553; M. T. Hemman et al., Nat. Genet., 2003, 33: 396-400); interfering RNAs that target oncogenes, such as Raf-1 (T. F. Lou et al., Oligonucleotides, 2003, 13: 313- 324), K-Ras (T. R. Brummelkamp et al., Cancer Cell, 2002, 2: 243-247), and erbB-2 (G. Yang et al., J. Biol. Chem., 2004, 279: 4339-4345);.

In certain embodiments, conjugates of anti-receptor tyrosine kinases antibodies comprise a nucleic acid therapeutic agent that is a ribozyme. As used herein, the term “ribozyme” refers to a catalytic RNA molecule that can cleave other RNA molecules in a target-specific marmer Ribozymes can be used to downregulate the expression of any undesirable products of genes of interest. Examples of ribozymes that can be used in the practice of the present invention include, but are not limited to, those specific for receptor tyrosine kinase mRNA.

In certain embodiments, entities or moieties within conjugates of the anti-receptor tyrosine kinases antibodies comprise a photosensitizer used in photodynamic therapy (PDT). In PDT, local or systemic administration of a photosensitizer to a patient is followed by irradiation with light that is absorbed by the photosensitizer in the tissue or organ to be treated. Light absorption by the photosensitizer generates reactive species (e.g., radicals) that are detrimental to cells. For maximal efficacy, a photosensitizer typically is in a form suitable for administration, and also in a form that can readily undergo cellular internalization at the target site, often with some degree of selectivity over normal tissues.

Conjugates of anti-receptor tyrosine kinases antibodies associated with a photosensitizer can be used as new delivery systems in PDT. In addition to reducing photosensitizer aggregation, delivery of photosensitizers according to the present invention exhibits other advantages such as increased specificity for target tissues/organ and cellular internalization of the photosensitizer.

Photosensitizers suitable for use in the present invention include any of a variety of synthetic and naturally occurring molecules that have photosensitizing properties useful in PDT. In certain embodiments, the absorption spectrum of the photosensitizer is in the visible range, typically between 350 nm and 1200 nm, preferably between 400 nm and 900 nm, e.g., between 600 nm and 900 nm. Suitable photosensitizers that can be coupled to toxins according to the present invention include, but are not limited to, porphyrins and porphyrin derivatives (e.g., chlorins, bacteriochlorins, isobacteriochlorins, phthalocyanines, and naphthalocyanines); metalloporphyrins, metallophthalocyanines, angelicins, chalcogenapyrrillium dyes, chlorophylls, coumarins, flavins and related compounds such as alloxazine and riboflavin, fullerenes, pheophorbides, pyropheophorbides, cyanines (e.g., merocyanine 540), pheophytins, sapphyrins, texaphyrins, purpurins, porphycenes, phenothiaziniums, methylene blue derivatives, naphthalimides, nile blue derivatives, quinones, perylenequinones (e.g., hypericins, hypocrellins, and cercosporins), psoralens, quinones, retinoids, rhodamines, thiophenes, verdins, xanthene dyes (e.g., eosins, erythrosins, rose bengals), dimeric and oligomeric forms of porphyrins, and prodrugs such as 5-aminolevulinic acid (R. W. Redmond and J. N. Gamlin, Photochem. Photobiol., 1999, 70: 391-475).

Exemplary photosensitizers suitable for use in the present invention include those described in U.S. Pat. Nos. 5,171,741; 5,171,749; 5,173,504; 5,308,608; 5,405,957; 5,512,675; 5,726,304; 5,831,088; 5,929,105; and 5,880,145 (the contents of each of which are incorporated herein by reference in their entirety).

In certain embodiments, conjugates of anti-receptor tyrosine kinase polyspecific antibodies comprise a radiosensitizer. As used herein, the term “radiosensitizer” refers to a molecule, compound or agent that makes tumor cells more sensitive to radiation therapy. Administration of a radiosensitizer to a patient receiving radiation therapy generally results in enhancement of the effects of radiation therapy. The advantage of coupling a radiosensitizer to a targeting entity (e.g., anti-receptor tyrosine kinases antibodies capable of targeting intratumoral receptor tyrosine kinases) is that the radiosensitize effects only on target cells. For ease of use, a radiosensitizer should also be able to find target cells even if it is administered systemically. However, currently available radiosensitizers are typically not selective for tumors, and they are distributed by diffusion in a mammalian body. Receptor tyrosine kinases antibody conjugates of the present invention can be used as a new delivery system for radiosensitizers.

A variety of radiosensitizers are known in the art. Examples of radiosensitizers suitable for use in the present invention include, but are not limited to, paclitaxel (TAXOL®), carboplatin, cisplatin, and oxaliplatin (Amorino et al., Radiat. Oncol. Investig., 1999, 7: 343-352; Choy, Oncology, 1999, 13: 22-38; Safran et al., Cancer Invest., 2001, 19: 1-7; Dionet et al., Anticancer Res., 2002, 22: 721-725; Cividalli et al., Radiat. Oncol. Biol. Phys., 2002, 52: 1092-1098); gemcitabine (Gemzar®) (Choy, Oncology, 2000, 14: 7-14; Mornex and Girard, Annals of Oncology, 2006, 17: 1743- 1747); etanidazole (Nitrolmidazole®) (Inanami et al., Int. J. Radiat. Biol., 2002, 78: 267- 274); misonidazole (Tamulevicius et al., Br. J. Radiology, 1981, 54: 318-324; Palcic et al., Radiat. Res., 1984, 100: 340-347), tirapazamine (Masunaga et al., Br. J. Radiol., 2006, 79: 991-998; Rischin et al., J. Clin. Oncol., 2001, 19: 535-542; Shulman et al., Int. J. Radiat. Oncol. Biol. Phys., 1999, 44: 349-353); and nucleic acid base derivatives, e.g., halogenated purines or pyrimidines, such as 5-fluorodeoxyuridine (Buchholz et al., Int. J. Radiat. Oncol. Biol. Phys., 1995, 32: 1053-1058).

In certain embodiments, conjugates of anti-receptor tyrosine kinases antibodies comprise a radioisotope. Examples of suitable radioisotopes include any β-, β- or γ-emitter, which, when localized at a tumor site, results in cell destruction (S. E. Order, “Analysis, Results, and Future Prospective of the Therapeutic Use of Radiolabeled Antibody in Cancer Therapy”, Monoclonal Antibodies for Cancer Detection and Therapy, R. W. Baldwin et al. (Eds.), Academic Press, 1985). Examples of such radioisotopes include, but are not limited to, iodine-131 (¹³¹I), iodine-125 (¹²⁵I), bismuth-212 (²¹²Bi), bismuth-213 (²¹³Bi), astatine-211 (²¹¹At), rhenium-186 (⁸⁶Re), rhenium-188 (¹⁸⁸Re), phosphorus-32 (³²P), yttrium-90 (⁹⁰Y), samarium-153 (¹⁵³Sm), and lutetium-177 (¹⁷⁷Lu).

In certain embodiments, conjugates of anti-receptor tyrosine kinases antibodies comprise a superantigen or biologically active portion thereof. Superantigens constitute a group of bacterial and viral proteins that are extremely efficient in activating a large fraction of the T-cell population. Superantigens bind directly to the major histocompatibility complex (MHC) without being processed. In fact, superantigens bind unprocessed outside the antigenbinding groove on the MHC class II molecules, thereby avoiding most of the polymorphism in the conventional peptide-binding site.

A superantigen-based tumor therapeutic approach has been developed for the treatment of solid tumors. In this approach, a targeting moiety (e.g., an anti-receptor tyrosine kinases antibody or antigen-binding fragment thereof) is conjugated to a superantigen, providing a targeted superantigen. If the antibody, or antibody fragment, recognizes a tumor-associated antigen, the targeted superantigen, bound to tumors cells, can trigger superantigen-activated cytotoxic T-cells to kill the tumor cells directly by superantigen-dependent cell mediated cytotoxicity. (See, e.g., Sogaard et al., (1996) “Antibody-targeted superantigens in cancer immunotherapy,” Immunotechnology, 2(3): 151-162, the entire contents of which are herein incorporated by reference.)

Examples of superantigens for use in embodiments of the invention include, fusion proteins with wild-type staphylococcal enterotoxin A (SEA) (Giantonio et al., J. Clin. Oncol., 1997, 15: 1994-2007; Alpaugh et a., Clin. Cancer Res., 1998, 4: 1903-1914; Cheng et al., J. Clin. Oncol., 2004, 22: 602-609; the entire contents of each of which are herein incorporated by reference); staphylococcal superantigens of the enterotoxin gene cluster (egc) (Terman et al., Clin. Chest Med., 2006, 27: 321-324, the entire contents of which are herein incorporated by reference), and staphylococcal enterotoxin B (SEB) (Perabo et al.,Int. J. Cancer, 2005, 115: 591-598, the entire contents of which are herein incorporated by reference). A superantigen, or a biologically active portion thereof, can be associated to anti-receptor tyrosine kinases antibodies to form a conjugate comprising the antibody and a superantigen. Additional examples of superantigens suitable for use in the present invention include, but are not limited to, staphylococcal enterotoxin E (SEE)), Streptococcus pyogenes exotoxin (SPE), Staphylococcus aureus toxic shock-syndrome toxin (TSST-1), streptococcal mitogenic exotoxin (SME), and streptococcal superantigen (SSA).

In certain embodiments, conjugates of the anti-receptor tyrosine kinases antibodies may be used in directed enzyme prodrug therapy. In a directed enzyme prodrug therapy approach, a directed/targeted enzyme and a prodrug are administered to a subject, wherein the targeted enzyme is specifically localized to a portion of the subject's body where it converts the prodrug into an active drug. The prodrug can be converted to an active drug in one step (by the targeted enzyme) or in more than one step. For example, the prodrug can be converted to a precursor of an active drug by the targeted enzyme. The precursor can then be converted into the active drug by, for example, the catalytic activity of one or more additional targeted enzymes, one or more non-targeted enzymes administered to the subject, one or more enzymes naturally present in the subject or at the target site in the subject (e.g., a protease, phosphatase, kinase or polymerase), by an agent that is administered to the subject, and/or by a chemical process that is not enzymatically catalyzed (e.g., oxidation, hydrolysis, isomerization, epimerization, etc.).

Some embodiments of the invention utilize antibody-directed enzyme prodrug therapy (ADEPT), wherein an anti-receptor tyrosine kinases antibody is linked to an enzyme and injected in a subject, resulting in selective binding of the enzyme to tumor-associated or metstatic receptor tyrosine kinases. Subsequently, a prodrug is administered to the subject. The prodrug is converted to its active form by the enzyme only within or nearby the cancer cells. Selectivity is achieved by the specificity of the anti-receptor tyrosine kinases antibody and by delaying prodrug administration until there is a large differential between cancer and normal tissue enzyme levels. Cancer cells may also be targeted with the genes encoding for prodrug activating enzymes. This approach has been called virus-directed enzyme prodrug therapy (VDEPT) or more generally GDEPT (gene-directed enzyme prodrug therapy, and has shown good results in laboratory systems. Other versions of directed enzyme prodrug therapy include PDEPT (polymer-directed enzyme prodrug therapy), LEAPT (lectin-directed enzyme-activated prodrug therapy), and CDEPT (clostridial-directed enzyme prodrug therapy).

Nonlimiting examples of enzyme/prodrug/active drug combinations suitable for use in the present invention are described, for example, in Bagshawe et al., Current Opinions in Immunology, 1999, 11: 579-583; Wilman, “Prodrugs in Cancer Therapy”, Biochemical Society Transactions, 14: 375-382, 615th Meeting, Belfast, 1986; Stella et al., “Prodrugs: A Chemical Approach To Targeted Drug Delivery”, in “Directed Drug Delivery”, Borchardt et al., (Eds), pp. 247-267 (Humana Press, 1985). Nonlimiting examples of enzyme/prodrug/active anti-cancer drug combinations are described, for example, in Rooseboom et al., Pharmacol. Reviews, 2004, 56: 53-102.

Examples of prodrug activating enzymes include, but are not limited to, nitroreductase, cytochrome P450, purine-nucleoside phosphorylase, thymidine kinase, alkaline phosphatase, β-glucuronidase, carboxypeptidase, penicillin amidase, β-lactamase, cytosine deaminase, and methionine γ-lyase.

Examples of anti-cancer drugs that can be formed in vivo by activation of a prodrug by a prodrug activating enzyme include, but are not limited to, 5-(aziridin-1-eel)- 4-hydroxyl-amino-2-nitro-benzamide, isophosphoramide mustard, phosphoramide mustard, 2-fluoroadenine, 6-methylpurine, ganciclovir-triphosphate nucleotide, etoposide, mitomycin C, p-[N,N-bis(2-chloroethyl)amino]phenol (POM), doxorubicin, oxazolidinone, 9-aminocamptothecin, mustard, methotrexate, benzoic acid mustard, adriamycin, daunomycin, carminomycin, bleomycins, esperamicins, melphalan, palytoxin, 4-desacetylvinblastine-3-carboxylic acid hydrazide, phenylenediamine mustard, 4′-carboxyphthalato(1,2-cyclohexane-diamine) platinum, taxol, 5-fluorouracil, methylselenol, and carbonothionic difluoride.

In certain embodiments, a therapeutic (e.g., anti-cancer) agent comprises a conjugate of one or more anti-receptor tyrosine kinases antibodies and an anti-angiogenic agent. Antiangiogenic agents suitable for use in the present invention include any molecule, compound, or factor that blocks, inhibits, slows down, or reduces the process of angiogenesis, or the process by which new blood vessels form by developing from preexisting vessels. Such a molecule, compound, or factor can block angiogenesis by blocking, inhibiting, slowing down, or reducing any of the steps involved in angiogenesis, including (but not limited to) steps of (1) dissolution of the membrane of the originating vessel, (2) migration and proliferation of endothelial cells, and (3) formation of new vasculature by migrating cells.

Examples of anti-angiogenic agents include, but are not limited to, bevacizumab (AVASTIN®), celecoxib (CELEBREX®), endostatin, thalidomide, EMD121974 (Cilengitide), TNP-470, squalamine, combretastatin A4, interferon-α, anti-VEGF antibody, SU5416, SU6668, PTK787/2K 22584, Marimistal, AG3340, COL-3, Neovastat, and BMS-275291.

### Administration

Receptor tyrosine kinase monoclonal antibodies and/or antibody polypeptides in accordance with the invention and pharmaceutical compositions of the present invention may be administered according to any appropriate route and regimen. In some embodiments, a route or regimen is one that has been correlated with a positive therapeutic benefit.

In some embodiments, the exact amount administered may vary from subject to subject, depending on one or more factors as is well known in the medical arts. Such factors may include, for example, one or more of species, age, general condition of the subject, the particular composition to be administered, its mode of administration, its mode of activity, the the severity of disease; the activity of the specific receptor tyrosine kinase antibody employed; the specific pharmaceutical composition administered; the half-life of the composition after administration; the age, body weight, general health, sex, and diet of the subject; the time of administration, route of administration, and rate of excretion of the specific compound employed; the duration of the treatment; drugs used in combination or coincidental with the specific compound employed and the like. Pharmaceutical compositions may be formulated in dosage unit form for ease of administration and uniformity of dosage. It will be understood, however, that the total daily usage of the compositions of the present invention will be decided by an attending physician within the scope of sound medical judgment.

Compositions of the present invention may be administered by any route, as will be appreciated by those skilled in the art. In some embodiments, compositions of the present invention are administered by oral (PO), intravenous (IV), intramuscular (IM), intra-arterial, intramedullary, intrathecal, subcutaneous (SQ), intraventricular, transdermal, interdermal, intradermal, rectal (PR), vaginal, intraperitoneal (IP), intragastric (IG), topical (e.g., by powders, ointments, creams, gels, lotions, and/or drops), mucosal, intranasal, buccal, enteral, vitreal, sublingual; by intratracheal instillation, bronchial instillation, and/or inhalation; as an oral spray, nasal spray, and/or aerosol, and/or through a portal vein catheter.

In specific embodiments, receptor tyrosine kinases antibodies in accordance with the present invention and/or pharmaceutical compositions thereof may be administered intravenously, for example, by intravenous infusion. In specific embodiments, receptor tyrosine kinases antibodies in accordance with the present invention and/or pharmaceutical compositions thereof may be administered by intramuscular injection. In specific embodiments, receptor tyrosine kinases antibodies in accordance with the present invention and/or pharmaceutical compositions thereof may be administered by intratumoural injection. In specific embodiments, receptor tyrosine kinases antibodies in accordance with the present invention and/or pharmaceutical compositions thereof may be administered by subcutaneous injection. In specific embodiments, receptor tyrosine kinases antibodies in accordance with the present invention and/or pharmaceutical compositions thereof may be administered via portal vein catheter. However, the invention encompasses the delivery of receptor tyrosine kinases antibodies in accordance with the present invention and/or pharmaceutical compositions thereof by any appropriate route taking into consideration likely advances in the sciences of drug delivery.

In certain embodiments, receptor tyrosine kinases antibodies in accordance with the present invention and/or pharmaceutical compositions thereof may be administered at dosage levels sufficient to deliver from about 0.001 mg/kg to about 100 mg/kg, from about 0.01 mg/kg to about 50 mg/kg, from about 0.1 mg/kg to about 40 mg/kg, from about 0.5 mg/kg to about 30 mg/kg, from about 0.01 mg/kg to about 10 mg/kg, from about 0.1 mg/kg to about 10 mg/kg, or from about 1 mg/kg to about 25 mg/kg of subject body weight per day to obtain the desired therapeutic effect. The desired dosage may be delivered more than three times per day, three times per day, two times per day, once per day, every other day, every third day, every week, every two weeks, every three weeks, every four weeks, every two months, every six months, or every twelve months. In certain embodiments, the desired dosage may be delivered using multiple administrations (e.g., two, three, four, five, six, seven, eight, nine, ten, eleven, twelve, thirteen, fourteen, or more administrations).

### Prophylactic Applications

In some embodiments, receptor tyrosine kinases antibodies in accordance with the invention may be utilized for prophylactic applications. In some embodiments, prophylactic applications involve systems and methods for preventing, inhibiting progression of, and/or delaying the onset of cancer or a neurodegenerative disorder, and/or any other receptor tyrosine kinases-associated condition in individuals susceptible to and/or displaying symptoms of cancer or a neurodegenerative disorder.

### Combination Therapy

It will be appreciated that receptor tyrosine kinases antibodies and therapeutically active conjugates thereof in accordance with the present invention and/or pharmaceutical compositions thereof can be employed in combination therapies to aid in diagnosis and/or treatment. “In combination” is not intended to imply that the agents must be administered at the same time and/or formulated for delivery together, although these methods of delivery are within the scope of the invention. Compositions can be administered concurrently with, prior to, or subsequent to, one or more other desired therapeutics or medical procedures. In will be appreciated that therapeutically active agents utilized in combination may be administered together in a single composition or administered separately in different compositions. In general, each agent will be administered at a dose and/or on a time schedule determined for that agent.

The particular combination of therapies (e.g., therapeutics or procedures) to employ in a combination regimen will take into account compatibility of the desired therapeutics and/or procedures and the desired therapeutic effect to be achieved. It will also be appreciated that pharmaceutical compositions of the anti-receptor tyrosine kinases antibodies disclosed herein can be employed in combination therapies (e.g., combination chemotherapeutic therapies), that is, the pharmaceutical compositions can be administered concurrently with, prior to, or subsequent to, one or more other desired therapeutic and/or chemotherapeutic procedures.

Anti-receptor tyrosine kinases antibodies, or a pharmaceutically acceptable composition thereof, may be administered in combination with chemotherapeutic agents to treat primary or metastatic cancer. In some embodiments, an active ingredient is a chemotherapeutic agent, such as, but not limited to, Adriamycin, dexamethasone, vincristine, cyclophosphamide, fluorouracil, topotecan, taxol, interferons, platinum derivatives, taxane (e.g., paclitaxel), vinca alkaloids (e.g., vinblastine), anthracyclines (e.g., doxorubicin), epipodophyllotoxins (e.g., etoposide), cisplatin, methotrexate, actinomycin D, actinomycin D, dolastatin 10, colchicine, emetine, trimetrexate, metoprine, cyclosporine, daunorubicin, teniposide, amphotericin, alkylating agents (e.g., chlorambucil), 5-fluorouracil, campthothecin, cisplatin, metronidazole, imatinib, Gleevec™, sunitinib and Sutent® and combinations thereof.

In certain embodiments, anti-receptor tyrosine kinases antibodies, conjugates thereof, or a pharmaceutically acceptable composition thereof, are administered in combination with an antiproliferative or chemotherapeutic agent selected from any one or more of Abarelix, aldesleukin, Aldesleukin, Alemtuzumab, Alitretinoin, Allopurinol, Altretamine, Amifostine, Anastrozole, Arsenic trioxide, Asparaginase, Azacitidine, BCG Live, Bevacuzimab, Avastin, Fluorouracil, Bexarotene, Bleomycin, Bortezomib, Busulfan, Calusterone, Capecitabine, Camptothecin, Carboplatin, Carmustine, Celecoxib, Cetuximab, Chlorambucil, Cisplatin, Cladribine, Clofarabine, Cyclophosphamide, Cytarabine, Dactinomycin, Darbepoetin alfa, Daunorubicin, Denileukin, Dexrazoxane, Docetaxel, Doxorubicin (neutral), Doxorubicin hydrochloride, Dromostanolone Propionate, Epirubicin, Epoetin alfa, Erlotinib, Estramustine, Etoposide Phosphate, Etoposide, Exemestane, Filgrastim, floxuridine fludarabine, Fulvestrant, Gefitinib, Gemcitabine, Gemtuzumab, Goserelin Acetate, Histrelin Acetate, Hydroxyurea, Ibritumomab, Idarubicin, Ifosfamide, Imatinib Mesylate, Interferon Alfa-2a, Interferon Alfa-2b, Irinotecan, Lenalidomide, Letrozole, Leucovorin, Leuprolide Acetate, Levamisole, Lomustine, Megestrol Acetate, Melphalan, Mercaptopurine, 6-MP, Mesna, Methotrexate, Methoxsalen, Mitomycin C, Mitotane, Mitoxantrone, Nandrolone, Nelarabine, Nofetumomab, Oprelvekin, Oxaliplatin, Paclitaxel, Palifermin, Pamidronate, Pegademase, Pegaspargase, Pegfilgrastim, Pemetrexed Disodium, Pentostatin, Pipobroman, Plicamycin, Porfimer Sodium, Procarbazine, Quinacrine, Rasburicase, Rituximab, Sargramostim, Sorafenib, Streptozocin, Sunitinib Maleate, Talc, Tamoxifen, Temozolomide, Teniposide, VM-26, Testolactone, Thioguanine, 6-TG, Thiotepa, Topotecan, Toremifene, Tositumomab, Trastuzumab, Tretinoin, ATRA, Uracil Mustard, Valrubicin, Vinblastine, Vincristine, Vinorelbine, Zoledronate, or Zoledronic acid.

The particular combination of therapies (e.g., Doxorubicin, ARN-509 and therapeutic antibodies to receptor tyrosine kinases, etc.) to employ in a combination regimen will generally take into account compatibility of the desired therapeutics and/or procedures and the desired therapeutic effect to be achieved. It will also be appreciated that the therapies and/or chemotherapeutics employed may achieve a desired effect for the same disorder (for example, an inventive antigen may be administered concurrently with another chemotherapeutic or neurological drug), or they may achieve different effects. It will be appreciated that the therapies employed may achieve a desired effect for the same purpose (for example, receptor tyrosine kinase-polyspecific antibodies useful for treating, preventing, and/or delaying the onset of cancer or a neurodegenerative disorder may be administered concurrently with another agent useful for treating, preventing, and/or delaying the onset of cancer or a neurodegenerative disorder), or they may achieve different effects (e.g., control of any adverse effects). The invention encompasses the delivery of pharmaceutical compositions in combination with agents that may improve their bioavailability, reduce and/or modify their metabolism, inhibit their excretion, and/or modify their distribution within the body.

In some embodiments, agents utilized in combination with be utilized at levels that do not exceed the levels at which they are utilized individually. In some embodiments, the levels utilized in combination will be lower than those utilized individually.

In some embodiments, combination therapy may involve administrations of a plurality of antibodies directed to a single epitope (e.g., a single conformational epitope). In some embodiments, combination therapy can comprise a plurality of antibodies that recognize distinct epitopes.

### Kits

The invention provides a variety of kits for conveniently and/or effectively carrying out methods in accordance with the present invention. Kits typically comprise one or more receptor tyrosine kinase-polyspecific antibodies or antibody polypeptides in accordance with the invention (e.g., 5A10). In some embodiments, kits comprise a collection of different receptor tyrosine kinase-polyspecific antibodies to be used for different purposes (e.g., diagnostics, treatment, and/or prophylaxis). Typically kits will comprise sufficient amounts of receptor tyrosine kinase-polyspecific antibodies to allow a user to perform multiple administrations to a subject(s) and/or to perform multiple experiments. In some embodiments, kits are supplied with or include one or more receptor tyrosine kinase-polyspecific antibodies that have been specified by the purchaser.

In certain embodiments, kits for use in accordance with the present invention may include one or more reference samples; instructions (e.g., for processing samples, for performing tests, for interpreting results, for solubilizing receptor tyrosine kinase-polyspecific antibodies, for storage of receptor tyrosine kinase-polyspecific antibodies, etc.); buffers; and/or other reagents necessary for performing tests. In certain embodiments kits can comprise panels of antibodies. Other components of kits may include cells, cell culture media, tissue, and/or tissue culture media.

In some embodiments, kits include a number of unit dosages of a pharmaceutical composition comprising receptor tyrosine kinases-polyspecific antibodies. A memory aid may be provided, for example in the form of numbers, letters, and/or other markings and/or with a calendar insert, designating the days/times in the treatment schedule in which dosages can be administered. Placebo dosages, and/or calcium dietary supplements, either in a form similar to or distinct from the dosages of the pharmaceutical compositions, may be included to provide a kit in which a dosage is taken every day.

Kits may comprise one or more vessels or containers so that certain of the individual components or reagents may be separately housed. Kits may comprise a means for enclosing the individual containers in relatively close confinement for commercial sale, e.g., a plastic box, in which instructions, packaging materials such as styrofoam, etc., may be enclosed.

In some embodiments, kits are used in the treatment, diagnosis, and/or prophylaxis of a subject suffering from and/or susceptible to cancer or a neurodegenerative disorder. In some embodiments, such kits comprise (i) at least one receptor tyrosine kinases-polyspecific antibodies antibody; (ii) a syringe, needle, applicator, etc. for administration of the at least one receptor tyrosine kinases-polyspecific antibody to a subject; and (iii) instructions for use.

These and other aspects of the present invention will be further appreciated upon consideration of the following Examples, which are intended to illustrate certain particular embodiments of the invention but are not intended to limit its scope, as defined by the claims.

### Examples

### General Details

### Example 1

### Production of ⁸⁹Zr-Labeled Monoclonal Receptor Tyrosine Kinase Antibodies

MEHD7945A (Genentech, San Francisco, California) was bridged to desferrioxamine (DFO) via an isothiourea linker and radiolabeled with 89Zr (t1/2˜3.27 d). Conjugation of MEHD7945A to DFO was achieved via a N-succinimidyl linkage with a mAb:DFO ratio of ˜1:4. Efficient labeling of MEHD7945A with 89Zr was obtained with excellent radiochemical yields (>80%) and purities (>99%). ⁸⁹Zr was produced as previously described (see Deri M A, Zeglis B M, Francesconi L C, Lewis J S. PET imaging with 89Zr: from radiochemistry to the clinic. Nucl Med Biol. 2013;40:3-14;and Holland J P, Sheh Y, Lewis J S. Standardized methods for the production of high specific-activity zirconium-89. Nucl Med Biol. 2009;36:729-739.). Radiolabeling of MEHD7945A-DFO proceeded similar to previous reported literature. The specific activity was determined to be ˜3 mCi/mg. (see Janjigian Y Y, Viola-Villegas N, Holland J P, et al. Monitoring afatinib treatment in HER2-positive gastric cancer with 18F-FDG and 89Zr-trastuzumab PET. J Nucl Med. 2013;54:936-943; and Holland J P, Caldas-Lopes E, Divilov V, et al. Measuring the pharmacodynamic effects of a novel Hsp90 inhibitor on HER2/neu expression in mice using Zr-DFO-trastuzumab. PLoS One. 2010;5:e8859).

### Example 2

### In vitro Analysis of ⁸⁹Zr-Labeled MEHD7945A Antibodies

In vitro experiments establishing immunoreactivity, protein binding and rate of internalization were conducted. Cells with variable levels of constitutive EGFR/HER3 expression were utilized. Criteria for sufficient immunoreactivity of >70% must be established to maintain functionality for the target. Immunoreactivity studies were performed according to the protocol by Lindmo et al. (see Lindmo T, Boven E, Cuttitta F, Fedorko J, Bunn P A, Jr. Determination of the immunoreactive fraction of radiolabeled monoclonal antibodies by linear extrapolation to binding at infinite antigen excess. J Immunol Methods. 1984;72:77-89.) to validate sufficient activity toward the two receptors. The final 89Zr-MEHD7945A construct proved to be robust, exhibiting an immunoreactivity of >98%. In vitro internalization assays using an EGFR(+) and HER3(+) cell line BxPC3 and EGFR/HER3 (−) cell line MIA Paca2 were performed to compare and demonstrate mode of uptake.

### Example 3

### In vivo Imaging Studies of ⁸⁹Zr-Labeled MEHD7945A Antibodies

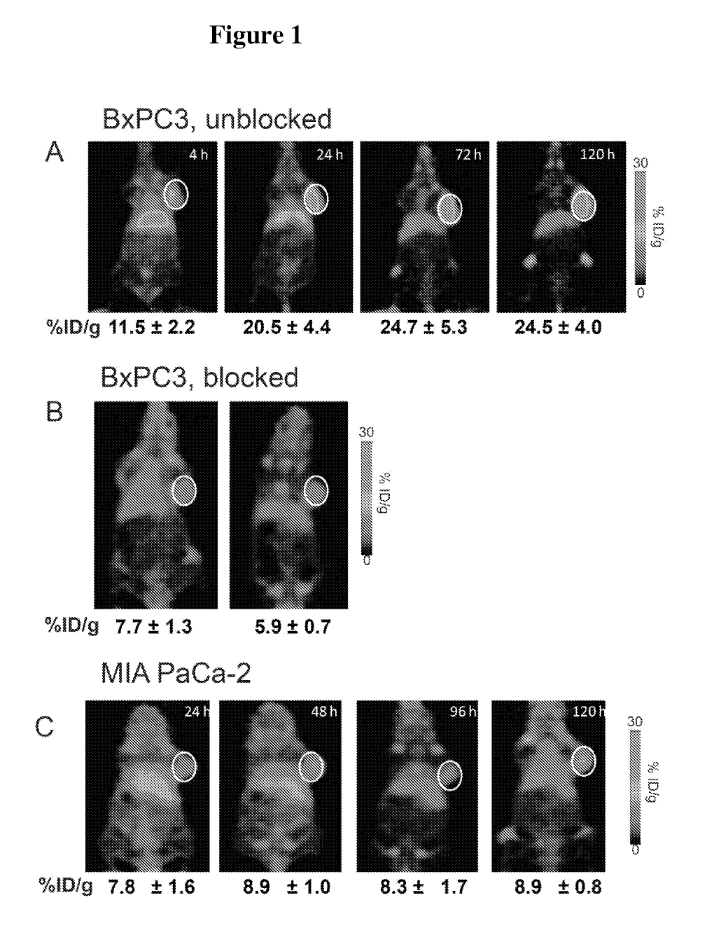

For in vivo experiments, tumor implants with positive or negative EGFR/HER3 expression were established in small animals. PET imaging and tissue distribution assays were conducted at several time points (i.e. 4, 24, 48, 96 120 hours where applicable) to evaluate overall distribution of tracer uptake in tumors and in normal tissues as depicted in FIG. 1. PET imaging was conducted on female SCID mice bearing subcutaneous BxPC3 xenografts implanted on the right shoulder at 4-120 hours post-tracer injection (p.i.). FIG. 1A depicts in vivo PET imaging studies demonstrated high 89Zr-MEHD7945A uptake in BxPC3 tumors where volumes-of-interest drawn on the tumor show incremental radiotracer uptake at 4 hours (11.5±2.2% ID/g), 24 hours (20.5±4.4% ID/g), which reached a plateau at 72 hours and 120 hours p.i (24.7±5.3% ID/g and 24.5±4.0% ID/g respectively). FIG. 1B shows that specificity was determined through competitive inhibition with 100-fold excess cold MEHD7945A co-administered with the tracer as demonstrated with a minimum two-fold lower tumor uptake (7.7±1.3% ID/g) obtained from the PET scans at 24 hour post-injection compared to the unblocked study Inhibition by addition of a blocking dose of cold MEHD7945A lowered the uptake of 89Zr-MEHD7945A in the same mouse model demonstrating specificity of the imaging probe. Furthermore, FIG. 1C demonstrates imaging EGFR/HER3(−) MIA PaCa-2 tumor implants with the same probe displayed stagnant uptake <10% ID/g at all timepoints (24-120 hours p.i.), possibly due to enhanced permeation and retention of the tumor's leaky vasculature.

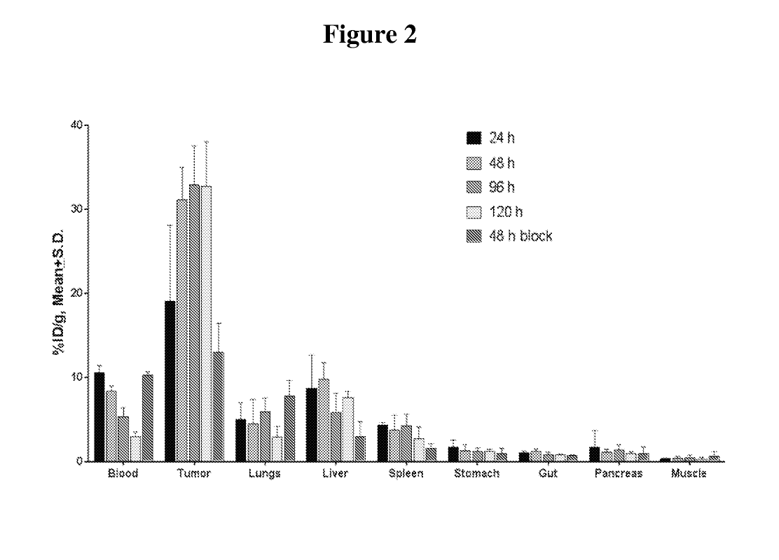

To determine overall tissue uptake of the imaging probe, distribution studies in parallel tumor models were performed at 24-120 hours p.i. in mice bearing EGFR/HER3(+) BxPC3 xenografts and are depicted in FIG. 2. Uptake in the tumor started to accumulate as early as 4 hours, reaching its peak at 48 hours p.i. while apparent tumor retention was observed as long as 120 hours p.i. From the tissue distribution, tumor uptake increased from 19.0±9.1% ID/g at 24 hours to 31.1±3.9% ID/g at 48 hours, 32.9±4.7% ID/g at 96 hours and finally 32.7±5.3% ID/g at 120 hours p.i. ⁸⁹Zr-MEHD7945A non-specifically binds to the liver; however, minimal non-specific uptake was observed in the pancreas and gastrointestinal tissues at all time points. Minimal accumulation was displayed in normal healthy tissues, particularly in the pancreas. From the tissue distribution, tumor-to-pancreas ratios clearly show excellent contrast across all time points (i.e. 31.1±9.6 at 48 hours). Blocking distribution studies proved the specificity of the probe with two-fold lower probe uptake found in the tumor with co-administration of 100-fold excess cold MEHD7945A in good agreement with the PET imaging results.

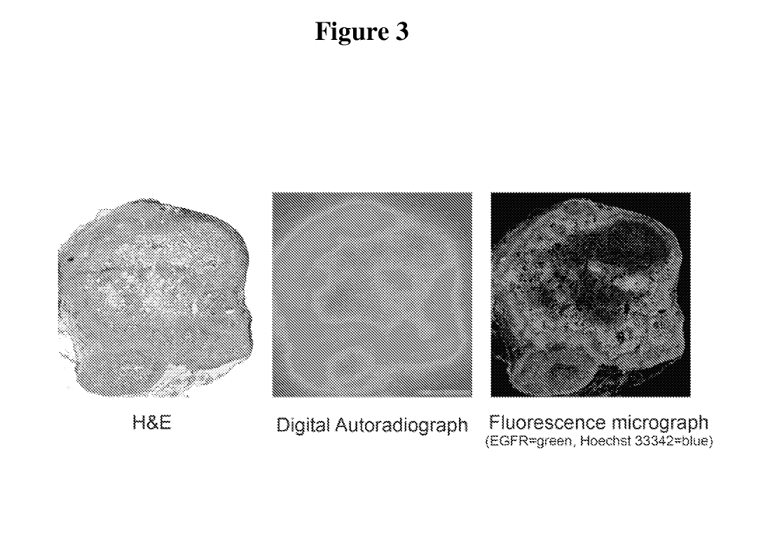

Ex vivo autoradiography and histology studies examined tissue localization of 89Zr-MEHD7945A versus EGFR and HER3. Autoradiography and fluorescence microscopy performed on ex vivo frozen tissue sections shows clear association with well-perfused regions containing receptor-expressing tumor cells. Minimal pooling of the probe in necrotic tumor regions was observed. FIG. 3 depicts the ex vivo histology and autoradiography, with H&E staining on the left, a digital autoradiograph at center, and an ex vivo fluorescence micrograph on the right, (green=EGFR, blue=Hoechst 33342). All data shown is from a single frozen section obtained from a subcutaneous BxPC3 murine xenograft model treated with ⁸⁹Zr-MEHD7945A.

### Example 4

### Imaging EGFR and HER3-Expressing Malignancies

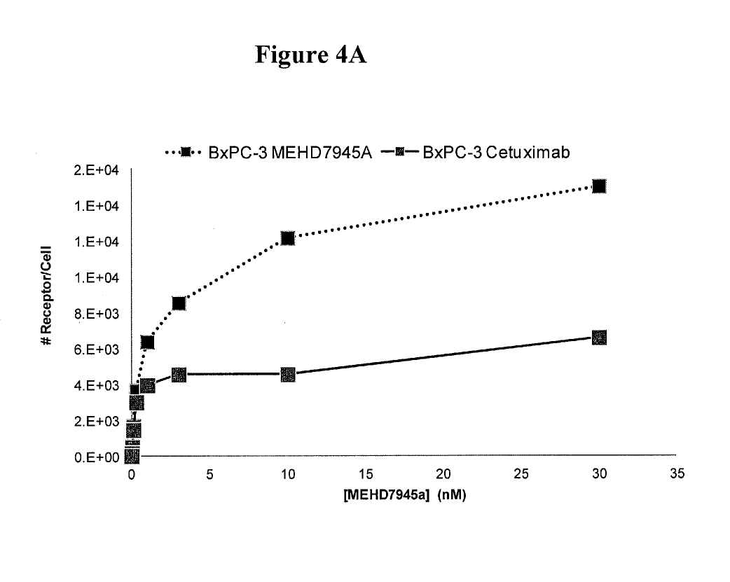

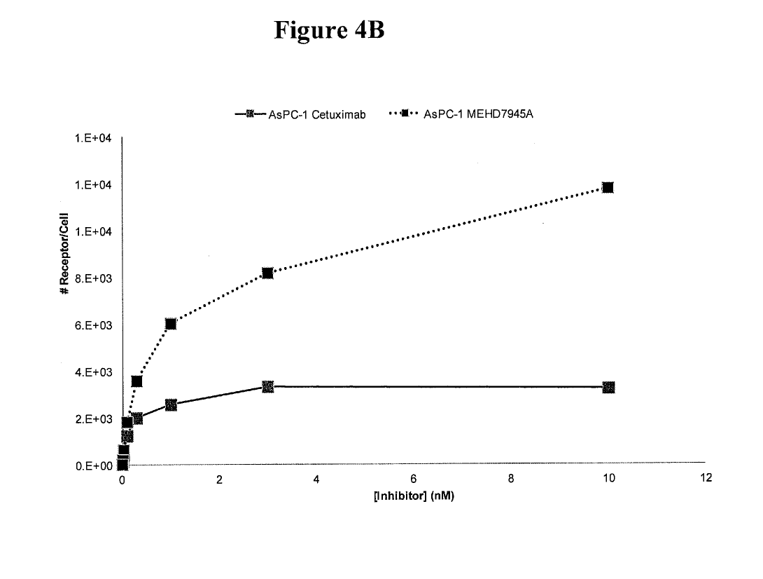

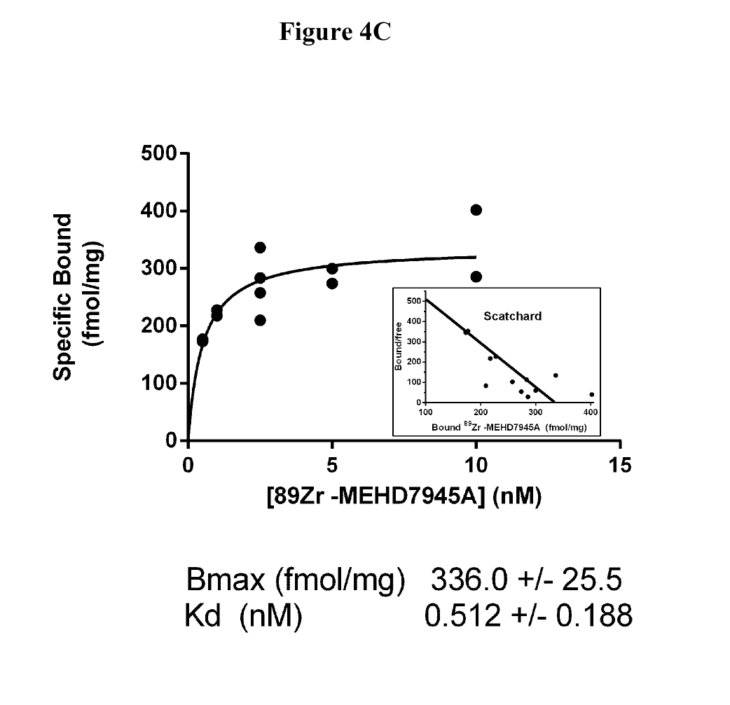

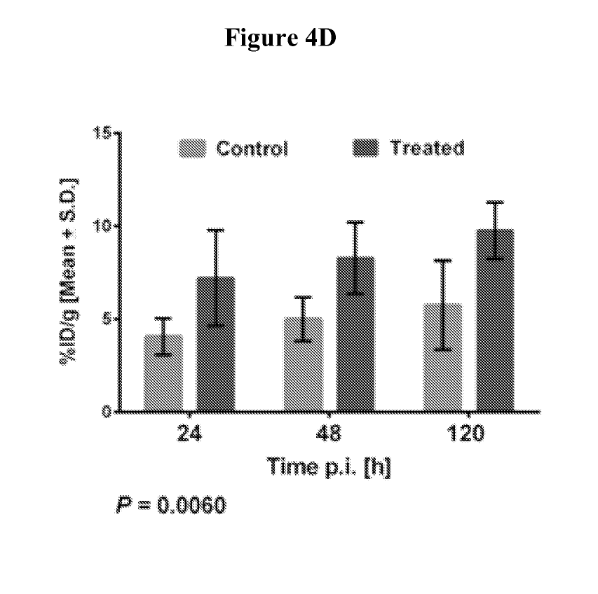

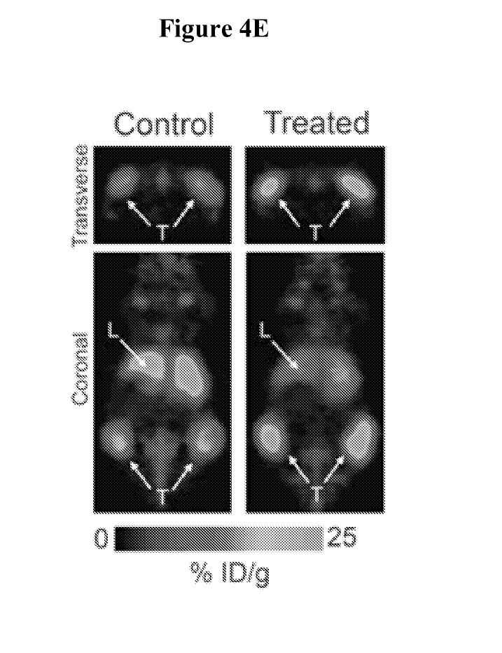

In vitro binding of ⁸⁹Zr-MEHD7945A was investigated in BxPC3 (KRAS-wt) and AsPC-1 (KRAS-mutant) cells. Two binding affinities were observed for ⁸⁹Zr-MEHD7945A in BxPC3 cells at 0.34 nM and 12.02 nM (FIG. 4A). Similar binding affinities were also observed for ⁸⁹Zr-MEHD7945A in AsPC-1 cells at 0.30 nM and 23.5 nM (FIG. 4B). In addition, ex vivo binding analysis was performed on 10 μM excised sections of AsPC-1 tumors. A non-linear regression analysis and Scatchard plot of ⁸⁹Zr-MEHD7945A in AsPC-1 tumors determined a K_{d}˜0.51±0.19 nM and a Bₘₐₓ of 336.0±25.5 fmol/mg (FIG. 4C).

To demonstrate that ⁸⁹Zr-MEHD7945A was able to measure pharmacological effects of treatment, mice bearing patient-derived triple negative breast cancer xenografts were dosed with GDC-0068, an Akt inhibitor, or vehicle. After a month, the tumors were imaged with ⁸⁹Zr-MEHD7945A. Compared to placebo-treated mice, ⁸⁹Zr-MEHD7945A detected a 1.5-fold increase in EGFR and HER3 expression (see FIG. 4D) from the regions-of-interest drawn on the PET scans of treated mice (FIG. 4E) (Tao et al., Sci. Signal, 2014, 7:318).

### Equivalents and Scope

Those skilled in the art will recognize, or be able to ascertain using no more than routine experimentation, many equivalents to the specific embodiments of the invention described herein. The scope of the present invention is not intended to be limited to the above Description. Likewise, those of ordinary skill in the art will readily appreciate that the foregoing represents merely certain preferred embodiments of the invention. Various changes and modifications to the procedures and compositions described above can be made without departing from the spirit or scope of the present invention, as set forth in the following claims.

In the claims articles such as “a”, “an” and “the” may mean one or more than one unless indicated to the contrary or otherwise evident from the context. Thus, for example, reference to “an antibody” includes a plurality of such antibodies, and reference to “the cell” includes reference to one or more cells known to those skilled in the art, and so forth. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are presenting, employed in, or otherwise relevant to a given product or process. Furthermore, it is to be understood that the invention encompasses all variations, combinations, and permutations in which one or more limitation, elements, clauses, descriptive terms, etc., from one or more of the listed claims is introduced into another claim. For example, any claim that is dependent on another claim can be modified to include one or more limitations found in any other claim that is dependent on the same base claim. Furthermore, where the claims recite a composition, it is to be understood that methods of using the composition for anyone of the purposes disclosed herein are included, and methods of making the composition according to any of the methods of making disclosed herein or other methods known in the art are included, unless otherwise indicated or unless it would be evident to one of ordinary skill in the art that a contradiction or inconsistency would arise.

Where elements are presented as lists, e.g., in Markush group format, it is to be understood that each subgroup of the elements is also disclosed, and any element(s) can be removed from the group. It should be understood that, in general, where the invention, or aspects of the invention, is/are referred to as comprising particular elements, features, etc., certain embodiments of the invention or aspects of the invention consist, or consist essentially of, such elements, features, etc. For purposes of simplicity those embodiments have not been specifically set forth in haec verba herein. It is noted that the term “comprising” is intended to be open and permits the inclusion of additional elements or steps.

Where ranges are given, endpoints are included. Furthermore, it is to be understood that unless otherwise indicated or otherwise evident from the context and understand of one of ordinary skill in the art, values that are expressed as ranges can assume any specific value or sub-range within the state ranges in different embodiments of the invention, to the tenth of the unit of the lower limit of the range, unless the context clearly dictates otherwise.

In addition, it is to be understood that any particular embodiment of the present invention that falls within the prior art may be explicitly excluded from any one or more of the claims. Since such embodiments are deemed to be known to one of ordinary skill in the art, they may be excluded even if the exclusion is not set forth explicitly herein. Any particular embodiment of the compositions of the invention can be excluded from any one or more claims, for any reason, whether or not related to the existence of prior art.

The publications discussed above and throughout the text are provided solely for their disclosure prior to the filing date of the present application. Nothing herein is to be construed as an admission that the inventors are not entitled to antedate such disclosure by virtue of prior disclosure.

What is claimed is:

1. An imaging method comprising
Administering to a subject suffering from or susceptible to a disease a poly-specific imaging agent comprising:
At least a first targeting moiety that interacts specifically with a first target marker;
At least a second targeting moiety that interacts specifically with a second target marker different from the first target marker; and
At least one associated detectable moiety, wherein
each of the first and second target markers is characterized in that its level of expression or activity is associated with a relevant disease state wherein each of the first and second target markers is characterized by correlation with the same relevant cancer state; and
Detecting the detectable moiety using an imaging modality.

2. The method of claim 1, wherein each of the first and second target markers is characterized by correlation with the same relevant disease state.

3. The method of claim 1, wherein disease state is selected from the group comprising:
neurodegenerative disorders, solid tumors, or cancer.

4. The method of claim 1, wherein the neurodegenerative disorder is selected from Alzheimer's Disease, Multiple Sclerosis, Huntington's Disease, Amyotrophic lateral sclerosis, Parkinson's Disease and muscular dystrophy.

5. The method of claim 1, wherein the cancer is selected from the group comprising: breast, pancreatic, non-small-cell lung, head and neck, anal and brain cancer.

6. The method of claim 1, wherein the imaging agent is bi-specific.

7. The method of claim 1, wherein the imaging agent comprises immunological moieties.

8. The method of claim 1, wherein the first targeting moiety is selected from the group comprising: immunoglobulins, antibodies, antibody fragments, peptides, and polypeptides.

9. The method of claim 1, wherein the first target marker is selected from the group comprising:
DNA, RNA, proteins, protein complexes, phosphorylated proteins, glycosylated proteins, folded proteins, and denatured proteins.

10. The method of claim 1, wherein the second targeting moiety is selected from the group comprising: immunoglobulins, antibodies, antibody fragments, peptides, and polypeptides.

11. The method of claim 1, wherein the second target marker is selected from the group comprising: DNA, RNA, proteins, protein complexes, phosphorylated proteins, glycosylated proteins, folded proteins, and denatured proteins.

12. The method of claim 1, wherein the first and second targeting moieties are simultaneously selected from the group comprising: immunoglobulins, antibodies, antibody fragments, peptides, and polypeptides.

13. The method of claim 1, wherein the first and second target markers are simultaneously selected from the group comprising: DNA, RNA, proteins, protein complexes, phosphorylated proteins, glycosylated proteins, folded proteins, and denatured proteins.

14. The method of claim 1, wherein the first and second target markers are simultaneously selected from the group comprising: Anaplastic lymphoma kinase, Alpha-fetoprotein (AFP), Beta-2-microglobulin (B2M), Beta-human chorionic gonadotropin (Beta-hCG), BCR-ABL, BRAF, CA15-3/CA27.29, CA19-9, CA-125, Calcitonin, Carcinoembryonic antigen (CEA), CD20, Chromogranin A (CgA), Cytokeratin, EGFR, Estrogen receptor (ER), progesterone receptor (PR), Fibrin/fibrinogen, HE4, HER2, Immunoglobulins, KIT, KRAS, Lactate dehydrogenase, Matrix metalloproteinases (MMP), Nuclear matrix protein 22, Prostate-specific antigen (PSA), Receptor Tyrosine kinases, Thyroglobulin, Urokinase plasminogen activator (uPA), and plasminogen activator inhibitor (PAI-1).

15. The method of claim 1, wherein the poly-specific imaging agent simultaneously targets at least two markers selected from the receptor tyrosine kinase protein family.

16. The method of claim 1, wherein the poly-specific imaging agent simultaneously targets at least two markers selected from the group comprising: EGFR, HER2, HER3, HER4, FGF1, FGF2, FGF3, FGF4, FGF5, FGF6, FGF7, FGF18, FGF21, VEGF-A, VEGF-B, VEGF-C, VEGF-D, PIGF, EphA1, EphA2, EphA3, EphA4, EphA5, EphA6, EphA7, EphA8, EphA9, EphA10, EphB1, EphB2, EphB3, EphB4, and EphB6.

17. The method of claim 1, wherein the poly-specific imaging agent simultaneously targets EGFR and HER3.

18. The method of claim 1, wherein the relevant disease state is a state of cancer responsiveness selected from the group comprising: responsive to therapy and resistant to resistant.

19. The method of claim 1, wherein the relevant disease state is a stage of cancer selected from the group comprising: stage 0, stage I, stage II, stage III, or stage IV.

20. The method of claim 1, wherein the imaging modality is selected from the group comprising, Positron Emission Tomography (PET), Single Photon Emission Tomography (SPECT), Computed Tomography (CT), Magnetic Resonance Imaging (MRI), Ultrasound Imaging (US), and Optical Imaging.

21. The method of claim 1, wherein the imaging modality is Positron Emission Tomography (PET).

22. The method of claim 1, wherein the detectable moiety is selected from the group comprising: a radiolabel, a fluorophore, a fluorochrome, an optical reporter, a magnetic reporter, an X-ray reporter, an ultrasound imaging reporter or a nanoparticle reporter.

23. The method of claim 1, wherein the detectable moiety is a radiolabel selected from the group comprising a radioisotopic element selected from the group consisting: of astatine, bismuth, carbon, copper, fluorine, gallium, indium, iodine, lutetium, nitrogen, oxygen, phosphorous, rhenium, rubidium, samarium, technetium, thallium, yttrium, and zirconium.

24. The method of claim 1, wherein the radiolabel is selected from the group comprising zirconium-89 (⁸⁹Zr), iodine-124 (¹²⁴I), iodine-131 (¹³¹I), iodine-125 (¹²⁵I), iodine-123 (¹²³I), bismuth-212 (²¹²Bi), bismuth-213 (²¹³Bi), astatine-221 (²¹¹At), copper-67 (⁶⁷Cu), copper-64 (⁶⁴Cu), rhenium-186 (¹⁸⁶_{Re}),rhenium-186 (¹⁸⁸Re), phosphorus-32 (³²P), samarium-153 (¹⁵³Sm), lutetium-177 (¹¹⁷Lu), (technetium-99m (^{99m}Tc), gallium-67 (⁶⁷Ga), indium-111 (¹¹¹In), thallium-201 (²⁰¹Tl) carbon-11, nitrogen-13 (¹³N), oxygen-15 (¹⁵O), fluorine-18 (¹⁸F), and rubidium-82 (⁸²Ru).

25. A poly-specific imaging agent comprising:
At least a first targeting moiety that interacts specifically with a first target marker;
At least a second targeting moiety that interacts specifically with a second target marker different from the first target marker; and
At least one associated detectable moiety.

26. A method for treating or reducing the risk of disease comprising: administering to a subject susceptible to the disease, disorder, or condition the agent of claim 25.

27. A kit for detecting the expression of target markers comprising the polyspecific imaging agent of any of claims 1-26.
